(19) United States
(12) Patent Application Publication
(10) Pub. No.: US 2014/0113342 A1
(43) Pub. Date: Apr. 24, 2014

(54) Production of 1,2-Propanediol in Cyanobacteria

(71) Applicant: Algenol Biofuels Inc., Fort Myers, FL (US)

(72) Inventors: Karl ZIEGLER, Zeuthen (DE); Christian WEISSERT, Berlin (DE); Ulf DUEHRING, Fredersdorf (DE); Jonathan Wong CHIN, Fort Myers, FL (US); Matthew Alexander ANDERSON, Estero, FL (US); Jianping CUI, Naples, FL (US); Matt SPIEKER, San Diego, CA (US)

(73) Assignee: Algenol Biofuels Inc., Fort Myers, FL (US)

(21) Appl. No.: 14/057,014

(22) Filed: Oct. 18, 2013

Related U.S. Application Data
(60) Provisional application No. 61/715,435, filed on Oct. 18, 2012.

(51) Int. Cl. C12P 7/18

(52) U.S. Cl. CPC C12P 7/18
USPC 435/158; 435/257.2

(57) ABSTRACT

Cyanobacterial host cells are modified to produce 1,2-propanediol.

### CROSS-REFERENCE TO RELATED APPLICATIONS

This application claims the benefit of U.S. Provisional Patent Application No. 61/715,435, filed Oct. 18, 2012, the disclosure of which is incorporated herein by reference.

### STATEMENT REGARDING FEDERALLY SPONSORED RESEARCH

Not Applicable.

### REFERENCE TO SEQUENCE LISTING

This application contains a sequence listing submitted by EFS-Web, thereby satisfying the requirements of 37 C.F.R. §§1.821-1.825. The Sequence Listing, created on Oct. 10, 2013, is named “1_{—}2-Propanediol_SEQ_LIST_US_ST25”, and is 160 KB in size.

### FIELD OF THE INVENTION

The present invention relates to cyanobacterial host cells which are modified to produce 1,2-propanediol.

### BACKGROUND OF THE INVENTION

Cyanobacteria (also known as “blue-green algae”) are small, mainly aquatic, prokaryotic cells that have the ability to perform oxygenic photosynthesis and make biomass and organic compounds from the input of light, nutrients, and CO₂. Cyanobacteria can be genetically enhanced to produce valuable products, such as biofuels, pharmaceuticals, nutrients, carotenoids, etc. For example, the transformation of the cyanobacterial genus Synechococcus with genes that encode specific enzymes that can produce ethanol for biofuel production has been described (U.S. Pat. Nos. 6,699,696 and 6,306,639, both to Woods et al.). The transformation of the cyanobacterial genus Synechocystis is described, for example, in PCT/EP2009/000892 and in PCT/EP2009/060526.

1,2-propanediol (also termed propylene glycol, propane-1,2-diol, 1,2-dihydroxypropane, and methylethylene glycol) is a three-carbon diol that is chiral with an asymmetric carbon at the 2-position. 1,2-propanediol is a colorless, viscous, water-miscible liquid. 1,2-propanediol, as a racemic mixture, is used in many industrial applications, including as a solvent in pharmaceuticals, as a de-icer for aircraft wings, and in deodorant sticks. 1,2-propanediol also has a relatively low human toxicity. The current commonly used pathway of production of 1,2-propanediol is through propylene from crude oil.

A metabolically engineered biosynthetic pathway for production of 1,2-propanediol in E. coli has been elucidated (FIG. 2; Altaras, N. E. et al., 1999, Applied and Environ. Biol. 65:1180-1185; and Altaras, N. E. et al., (2000) Biotech. Progress 16:940-946). The pathway can produce either S-1,2-propanediol or R-1,2-propanediol, depending on the chosen enzymes.

Current methods of producing 1,2-propanediol require the input of an organic carbon source, such as fossil fuel or sugar. What is needed is a method of producing these compounds from CO₂ as the input carbon source, rather than from fossil fuels or from other organic starting materials.

### SUMMARY OF THE INVENTION

In an aspect of the invention, a genetically enhanced nucleic acid sequence for the production of 1,2-propanediol in cyanobacteria is provided, having at least one promoter capable of regulating gene expression in cyanobacteria; and the following genes: gldA, fucO, mgsA, and optionally yqhD. The nucleic acid sequence can be capable of replicating in a cyanobacterial cell. At least one of the genes can be located on an exogenously or endogenously derived plasmid, or on the cyanobacterial chromosome. The promoter can be, for example, Psrp (such as SEQ ID NO: 1), PnblA₇₁₂₀ (such as SEQ ID NO: 2), PrbcL₆₈₀₃ or derivatives (such as SEQ ID NO: 3, 4, 5, or 6), PsmtA₇₀₀₂ (such as SEQ ID NO: 7), ziaR-PziaA₆₈₀₃ (such as SEQ ID NO: 8), or PpetJ (such as SEQ ID NO: 9). The gldA gene can have at least 98% identity to SEQ ID NO: 11. The GldA polypeptide can have at least 98% identity to SEQ ID NO: 12. The fucO gene can have at least 98% identity to SEQ ID NO: 13. The FucO polypeptide can have at least 98% identity to SEQ ID NO: 14. The mgsA gene can have at least 98% identity to SEQ ID NO: 15. The MgsA polypeptide can have at least 98% identity to SEQ ID NO: 16. The yqhD gene can have at least 98% identity to SEQ ID NO: 17. The YqhD polypeptide can have at least 98% identity to SEQ ID NO: 18. In another aspect of the invention, a genetically modified cyanobacterial cell having a heterologous nucleic acid sequence of any one of the above sequences is provided, where the cell can produce 1,2-propanediol.

In another aspect of the invention, a genetically enhanced cyanobacterial cell is provided, having a gldA gene, a fucO gene, an mgsA gene, and optionally a yqhD gene, wherein the cyanobacterial cell produces 1,2-propanediol. The genes can be located together under the control of one promoter, or at least one of the genes can be present in another location in the cell. The cyanobacterium can be, for example, Synechocystis sp. PCC 6803, or Synechococcus sp. PCC 7002.

In yet another aspect of the invention, a method of producing 1,2-propanediol in a cyanobacterial cell is provided, by introducing a nucleic acid sequence having a gene encoding a GldA enzyme, a gene encoding a FucO enzyme, a gene encoding an MgsA enzyme, and optionally a gene encoding a YqhD enzyme into a cyanobacterial cell; and then culturing the cyanobacterial cell under conditions to produce 1,2-propanediol.

In another aspect of the invention, a method of producing 1,2-propanediol in a cyanobacterial cell is provided, by transforming the cell with an mgsA gene, a gene encoding an enzyme capable of converting methylglyoxal to lactaldehyde, and a gene encoding an enzyme capable of converting lactaldehyde to 1,2-propanediol. The gene encoding the enzyme capable of converting methylglyoxal to lactaldehyde can be selected, for example, from GldA, SynADH, and SynAKR. The gene encoding the enzyme capable of converting lactaldehyde to 1,2-propanediol can be selected, for example, from FucO and GldA. Each of the inserted genes can be under the control of separate promoters, or they can be under the control of one promoter. In a further embodiment, the SynAKR gene has a sequence of SEQ ID NO: 19. In another embodiment, the SynAKR protein is SEQ ID NO: 20. In an embodiment, the SynADH gene has a sequence of SEQ ID NO: 21. In another embodiment, the SynADH protein is SEQ ID NO: 22.

### BRIEF DESCRIPTION OF THE DRAWINGS

FIG. 1 is a diagram of one biosynthetic pathway used to produce 1,2-propanediol from the central carbon metabolites pyruvate and glycerone phosphate (DHAP). These metabolites can be produced through photosynthetic and gluconeogenic pathways using CO₂ as the input carbon source in cyanobacteria. As shown in the figure, the pathway involves the intermediate compounds glycerone phosphate, methylglyoxal, acetol, and 2-hydroxypropionaldehyde (“lactaldehyde”).

FIG. 2 is another diagram of a biosynthetic pathway that can be used to produce 1,2-propanediol. In this pathway, either the S- or the R-form of 1,2-propanediol can be formed. The pathway diagram is taken from Alteras et al., 1999.

FIG. 3 is another diagram of a biosynthetic pathway that can be used to produce 1,2-propanediol. Three possible alternative enzymes for the conversion of methylglyoxal to lactaldehyde are shown. Two possible alternative enzymes for the conversion of lactaldehyde to 1,2-propanediol are also shown. Further, the role of NADP and NADPH are also indicated.

FIG. 4 is a map of two gene cassettes (#728, #729) for transformation to cyanobacteria. Both of the gene cassettes encode the enzymes MgsA and synADH. The promoters, terminators, and relevant restriction sites are indicated.

FIG. 5 is a map of two gene cassettes (#747, #748) for transformation to cyanobacteria. Both of the gene cassettes encode the enzymes GldA, MgsA, and synADH deg, but different promoters are used to control the expression of each of the genes. The promoters, terminators, and relevant restriction sites are indicated.

FIG. 6 is a map of two gene cassettes (#749, #750) for transformation to cyanobacteria. Both of the gene cassettes encode the enzymes FucO, MgsA, and synADH deg., but different promoters are used to control the expression of each of the genes, as indicated. The terminator sequences and relevant restriction sites are also indicated.

FIG. 7 is a map of two gene cassettes (#767, #768) for transformation to cyanobacteria. Both of the gene cassettes encode the enzymes FucO, MgsA, and Syn7002AKR, but different promoters are used to control the expression of each of the genes, as indicated. The terminator sequences and relevant restriction sites are also indicated.

FIG. 8 is a map of two gene cassettes (#769, #770) for transformation to cyanobacteria. Both of the gene cassettes encode the enzymes GldA, MgsA, and Syn7002AKR, but different promoters are used to control the expression of each of the genes, as indicated. The terminator sequences and relevant restriction sites are also indicated.

FIG. 9 is a bar graph showing the in vitro MgsA and GldA/SynADH activity in Synechocystis PCC 6803 transformed with plasmids containing the #747 or #748 cassette, as indicated. The enzyme activity is measured in nmol per mg protein per minute.

FIG. 10 is a bar graph showing the GldA/SynADH in vitro activity in Synechocystis PCC 6803 transformed with plasmids containing the #747 or #748 cassette, as indicated.

FIG. 11 is a bar graph showing the production of hydroxyacetone and 1,2-propanediol in Synechocystis PCC 6803 transformed with plasmids containing the #749 or #750 cassette, as indicated.

FIG. 12 is a bar graph showing the in vitro activity of the enzyme MgsA for Synechocystis PCC 6803 cells transformed with plasmids containing either the #767-#768 constructs (FucO, MgsA, AKR) at day 2, 5, and 7, or the #769 construct (GldA, MgsA, AKR) at days 5 and 7. The enzyme activity is measured in nmol per mg protein per minute.

FIG. 13 is a bar graph showing the in vitro activity of 1) a combination of MgsA+SynAKR+FucO; 2) a combination of SynAKR+FucO; or 3) FucO activity alone. Synechocystis PCC 6803 cells were transformed with plasmids containing the #769 construct (GldA, MgsA, AKR). The cultures were measured at day 5 and day 7. The enzyme activity is measured in nmol per mg protein per minute.

FIG. 14 is a bar graph showing the in vitro activity of 1) a combination of MgsA+SynAKR+FucO; 2) a combination of SynAKR+FucO; or 3) FucO activity alone. Synechocystis PCC 6803 cells were transformed with plasmids containing the #767 or #768 construct (FucO, MgsA, and AKR) The cultures were measured at day 2, day 5, and day 7. The enzyme activity is measured in nmol per mg protein per minute.

FIG. 15 is a linear diagram of the genes and relevant features in the broad host range RSF1010-derivative plasmid pSL1211, which was used as the basis for the expression vectors described herein. Relevant restriction sites and terminator regions (TT) are indicated.

FIG. 16 is a linearized map of the pSL1211-derived plasmid (“pABb”) that was used as the framework plasmid for the insertion of the polycistronic propanediol genes described in Examples 10-12. The promoter, terminator (TT), and ribosomal binding site (RBS) are indicated.

FIG. 17 is a linearized map of the “GYFM” fragment that was inserted into plasmid pABb (FIG. 16) to create pAB1025 in order to produce 1,2-propanediol as described in Examples 10-12. The relevant restriction sites used for cloning are indicated.

FIG. 18 is a graph confirming the production of 1,2-propanediol in Synechococcus sp. PCC 7002. The graph represents a chromatographic trace of a 20× concentrated methanol/phosphate extract from a culture of PCC 7002 harboring the plasmid pAB1025. The trace was produced from a separation of 1,2 propanediol using gas chromatography and peaks were identified using mass spectroscopy. The peak at retention time 4.9 minutes was identified as 1,2 propanediol. This peak was not present in wild type Synechococcus sp. PCC 7002.

FIG. 19 is a graph confirming the production of 1,2-propanediol in Synechocystis sp. PCC 6803. The graph represents a chromatographic trace of a 15× concentrated methanol/phosphate extract from Synechocystis PCC 6803 harboring the plasmid pAB1025. The trace was produced from a separation of 1,2-propanediol using gas chromatography. The peaks were identified using mass spectroscopy. The peak having a retention time of 4.9 minutes was identified as 1,2-propanediol. This peak was not present in wild type Synechocystis sp. PCC 6803.

### DETAILED DESCRIPTION

Cyanobacterial host cells can be genetically enhanced in order to produce various valuable chemical products, such as 1,2-propanediol. In an embodiment, genes involved in the biosynthetic pathways for 1,2-propanediol production can be transferred to a cyanobacterial host cell. The inserted heterologous genes can be present on extrachromosomal plasmids, or they can be present on the cyanobacterial chromosome. The cyanobacterial cells are then cultured following general cyanobacterial methods, and the propanediol is removed at the appropriate time. The production of 1,2-propanediol in cyanobacteria rather than by use of chemical means allows the compounds to be produced from carbon dioxide as the initial carbon source, rather than from crude oil or other organic carbon sources.

Aspects of the invention utilize techniques and methods common to the fields of molecular biology, microbiology and cell culture. Useful laboratory references for these types of methodologies are readily available to those skilled in the art. See, for example, Molecular Cloning: A Laboratory Manual (Third Edition), Sambrook, J., et al. (2001) Cold Spring Harbor Laboratory Press; Current Protocols in Microbiology (2007) Edited by Coico, R, et al., John Wiley and Sons, Inc.; The Molecular Biology of Cyanobacteria (1994) Donald Bryant (Ed.), Springer Netherlands; Handbook Of Microalgal Culture Biotechnology And Applied Phycology (2003) Richmond, A.; (ed.), Blackwell Publishing; and “The cyanobacteria, molecular Biology, Genomics and Evolution”, Edited by Antonia Herrero and Enrique Flores, Caister Academic Press, Norfolk, UK, 2008.

All publications and patent applications mentioned in this specification are herein incorporated by reference to the same extent as if each individual publication or patent application was specifically and individually indicated to be incorporated by reference.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

The term “about” is used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) by a variance of 20%.

The term “Cyanobacterium” refers to a member from the group of photoautotrophic prokaryotic microorganisms which can utilize solar energy and fix carbon dioxide. Cyanobacteria are also referred to as blue-green algae.

The terms “host cell” and “recombinant host cell” are intended to include a cell suitable for metabolic manipulation, e.g., which can incorporate heterologous polynucleotide sequences, e.g., which can be transformed. The term is intended to include progeny of the cell originally transformed. In particular embodiments, the cell is a prokaryotic cell, e.g., a cyanobacterial cell. The term recombinant host cell is intended to include a cell that has already been selected or engineered to have certain desirable properties and to be suitable for further enhancement using the compositions and methods of the invention.

“Competent to express” refers to a host cell that provides a sufficient cellular environment for expression of endogenous and/or exogenous polynucleotides.

As used herein, the term “genetically enhanced” refers to any change in the endogenous genome of a wild type cell or to the addition of non-endogenous genetic code to a wild type cell, e.g., the introduction of a heterologous gene. More specifically, such changes are made by the hand of man through the use of recombinant DNA technology or mutagenesis. The changes can involve protein coding sequences or non-protein coding sequences such as regulatory sequences as promoters or enhancers.

The terms “Polynucleotide” and “nucleic acid” refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G, C), this also includes an RNA sequence (i.e., A, U, G, C) in which “U” replaces “T.”

The nucleic acids may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, alkylators, intercalators, pendent moieties, modified linkages, and chelators. Also included are synthetic molecules that mimic polynucleotides in their ability to bind to a designated sequence via hydrogen bonding and other chemical interactions.

A “promoter” is a nucleic acid control sequence that directs transcription of an associated polynucleotide, which may be a heterologous polynucleotide or a native polynucleotide. A promoter includes nucleic acid sequences near the start site of transcription, such as a polymerase binding site. The promoter also optionally includes distal enhancer or repressor elements which can be located as much as several thousand base pairs from the start site of transcription. In one embodiment, the transcriptional control of a promoter results in an increase in expression of the gene of interest. In another embodiment, a promoter is placed 5′ to the gene of interest.

A promoter can be used to replace the natural promoter, or can be used in addition to the natural promoter. A promoter can be endogenous with regard to the host cell in which it is used or it can be a heterologous polynucleotide sequence introduced into the host cell, e.g., exogenous with regard to the host cell in which it is used. A promoter can also be endogenous with regard to the host cell, but derived from a different original gene. In an embodiment, the promoter is a constitutive promoter. In another embodiment, the promoter is inducible, meaning that certain exogenous stimuli (e.g., nutrient starvation, heat shock, mechanical stress, light exposure, etc.) will induce the promoter leading to the transcription of the gene.

In one aspect the invention also provides nucleic acids which are at least 60%, 70%, 80% 90%, 95%, 97%, 98%, 99%, or 99.5% identical to the nucleic acids disclosed herein.

The term “nucleic acid” (also referred to as polynucleotide) is also intended to include nucleic acid molecules having an open reading frame encoding a polypeptide, and can further include non-coding regulatory sequences and introns. In addition, the terms are intended to include one or more genes that map to a functional locus. In addition, the terms are intended to include a specific gene for a selected purpose. The gene can be endogenous to the host cell or can be recombinantly introduced into the host cell.

The percentage of identity of two nucleic acid sequences or two amino acid sequences can be determined using the algorithm of Thompson et al. (CLUSTALW, 1994, Nucleic Acids Research 22: 4673-4680). A nucleotide sequence or an amino acid sequence can also be used as a so-called “query sequence” to perform a search against public nucleic acid or protein sequence databases in order, for example, to identify further unknown homologous promoters, which can also be used in embodiments of this invention. In addition, any nucleic acid sequences or protein sequences disclosed in this patent application can also be used as a “query sequence” in order to identify yet unknown sequences in public databases, which can encode for example new enzymes, which could be useful in this invention. Such searches can be performed using the algorithm of Karlin and Altschul (1990, Proceedings of the National Academy of Sciences U.S.A. 87: 2,264 to 2,268), modified as in Karlin and Altschul (1993, Proceedings of the National Academy of Sciences U.S.A. 90: 5,873 to 5,877). Such an algorithm is incorporated in the NBLAST and XBLAST programs of Altschul et al. (1990, Journal of Molecular Biology 215: 403 to 410). Suitable parameters for these database searches with these programs are, for example, a score of 100 and a word length of 12 for BLAST nucleotide searches as performed with the NBLAST program. BLAST protein searches are performed with the XBLAST program with a score of 50 and a word length of 3. Where gaps exist between two sequences, gapped BLAST is utilized as described in Altschul et al. (1997, Nucleic Acids Research, 25: 3,389 to 3,402).

“Recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. In an embodiment, the recombinant polynucleotide can be located on an extrachromosomal plasmid. In another embodiment, the recombinant nucleic acid can be located on the cyanobacterial chromosome. A host cell that comprises the recombinant polynucleotide is referred to as a “recombinant host cell” or a “recombinant bacterium” or a “recombinant cyanobacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see Watson et al., “Molecular Biology of the Gene,” pages 313-327, The Benjamin/Cummings Publishing Co. 4th ed. (1987).

The term “non-homologous or random integration” refers to any process by which DNA is integrated into the genome that does not involve homologous recombination. It appears to be a random process in which incorporation can occur at any of a large number of genomic locations.

The term “expressed endogenously” refers to polynucleotides that are native to the host cell and are naturally expressed in the host cell.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide. The nucleotide sequence of interest is linked to the regulatory sequence(s) in a manner which allows for regulation of expression (e.g., enhanced, increased, constitutive, basal, attenuated, decreased or repressed expression) of the nucleotide sequence and expression of a gene product encoded by the nucleotide sequence (e.g., when the recombinant nucleic acid molecule is included in a recombinant vector, as defined herein, and is introduced into a microorganism).

The term “vector” as used herein is intended to refer to a nucleic acid molecule capable of transporting another nucleic acid to which it has been linked. One type of vector is a “plasmid,” which generally refers to a circular double stranded DNA molecule into which additional DNA segments may be ligated, but also includes linear double-stranded molecules such as those resulting from amplification by the polymerase chain reaction (PCR) or from treatment of a circular plasmid with a restriction enzyme.

Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

In an embodiment, the RSF1010 vector, originally derived from E. coli, is used as a base plasmid for expression of the propanediol genes in Cyanobacteria. This vector appears to be relatively stable and can exist in the cell at a copy number of about 15-20 per cell.

Other plasmids, such as plasmids derived from an endogenous vector of the host cell strain or another cyanobacterial cell, may also be used. An “endogenous vector” or “endogenous plasmid” refers to an extrachromosomal, circular nucleic acid molecule that is derived from the host cell organism.

The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). The recombinant nucleic acid molecule (e.g., a recombinant DNA molecule) can also refer to a nucleic acid that originated in a different location on the DNA, or from a different organism.

The term “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence.

The term “endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “nucleic acid fragment” will be understood to mean a nucleotide sequence of reduced length relative to the reference nucleic acid and comprising, over the common portion, a nucleotide sequence substantially identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least about 6 to about 2200 or more consecutive nucleotides of a polynucleotide according to the invention.

The term “open reading frame,” abbreviated as “ORF,” refers to a length of nucleic acid sequence, either DNA, cDNA or RNA, that comprises a translation start signal or initiation codon, such as an ATG or AUG, and a termination codon and can be potentially translated into a polypeptide sequence.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “downstream” refers to a nucleotide sequence that is located 3′ to reference nucleotide sequence. In particular, downstream nucleotide sequences generally relate to sequences that follow the starting point of transcription. For example, the translation initiation codon of a gene is located downstream of the start site of transcription.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, “substantially similar” refers to nucleic acid fragments wherein changes in one or more nucleotide bases results in substitution of one or more amino acids, but do not affect the functional properties of the protein encoded by the DNA sequence.

The term “substantially similar” also refers to modifications of the nucleic acid fragments, such as the deletion or insertion of one or more nucleotide bases that do not substantially affect the functional properties of the resulting transcript.

The terms “restriction endonuclease” and “restriction enzyme” refer to an enzyme that binds and cuts within a specific nucleotide sequence within double stranded DNA.

The term “expression” as used herein refers to the transcription and stable accumulation mRNA derived from a nucleic acid or polynucleotide. Expression may also refer to translation of mRNA into a protein or polypeptide.

The term “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

The term “polymerase chain reaction,” also termed “PCR,” refers to an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

An “expression cassette” or “expression construct” refers to a series of polynucleotide elements that permit transcription of a gene in a host cell. Typically, the expression cassette includes a promoter and one or more heterologous or native polynucleotide sequences that are transcribed. Expression cassettes or constructs may also include, e.g., transcription termination signals, polyadenylation signals, and enhancer elements.

The term “codon” refers to a triplet of nucleotides coding for a single amino acid.

The term “codon-anticodon recognition” refers to the interaction between a codon on an mRNA molecule and the corresponding anticodon on a tRNA molecule.

The term “codon bias” refers to the fact that not all codons are used equally frequently in the genes of a particular organism.

The term “codon optimization” refers to the modification of at least some of the codons present in a heterologous gene sequence from a triplet code that is not generally used in the host organism to a triplet code that is more common in the particular host organism. This can result in a higher expression level of the gene of interest.

The expression constructs can be designed taking into account such properties as codon usage frequencies of the organism in which the recombinant genes are to be expressed. Codon usage frequencies can be determined using known methods (see, e.g., Nakamura et al. Nucl. Acids Res. 28:292, 2000). Codon usage frequency tables, including those for cyanobacteria, are also available in the art (e.g., in codon usage databases of the Department of Plant Genome Research, Kazusa DNA Research Institute (www.kazusa.or.jp/codon).

The term “transformation” is used herein to mean the insertion of heterologous genetic material into the host cell. Typically, the genetic material is DNA on a plasmid vector, but other means can also be employed. General transformation methods and selectable markers for bacteria and cyanobacteria are known in the art (Wirth, Mol Gen Genet. 216:175-177 (1989); Koksharova, Appl Microbiol Biotechnol 58:123-137 (2002); Sambrook et al, supra).

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, spectinomycin, kanamycin, hygromycin, and the like.

A “polypeptide” is a polymeric compound comprised of covalently linked amino acid residues. A “protein” is a polypeptide that performs a structural or functional role in a living cell.

The invention also provides amino acid sequences of the enzymes involved in 1,2-propanediol formation, which are at least 60%, 70%, 80%, 85%, 90%, 95%, 97%, 98%, 99%, or 99.5% identical to the amino acid sequences disclosed herein.

The EC numbers cited throughout this patent application are enzyme commission numbers. This is a numerical classification scheme for enzymes based on the chemical reactions which are catalyzed by the enzymes.

A “heterologous gene” refers to a gene that is not naturally present in the cell. Similarly, the term “heterologous nucleic acid” refers to a nucleic acid sequence that is not normally present in the cell.

A “heterologous protein” refers to a protein not naturally produced in the cell.

An “isolated polypeptide” or “isolated protein” is a polypeptide or protein that is substantially free of those compounds that are normally associated therewith in its natural state (e.g., other proteins or polypeptides, nucleic acids, carbohydrates, lipids).

The term “polypeptide fragment” of a polypeptide refers to a polypeptide whose amino acid sequence is shorter than that of the reference polypeptide. Such fragments of a polypeptide according to the invention may have a length of at least about 2 to about 750 or more amino acids.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements.

### Preparation of Recombinant Vectors for Genetic Modification of Cyanobacteria

Cyanobacteria can be modified to add enzymatic pathways of interest as shown herein in order to produce 1,2-propanediol. The DNA sequences encoding the genes described herein can be amplified by polymerase chain reaction (PCR) using specific primers. The amplified PCR fragments can be digested with the appropriate restriction enzymes and can then be cloned into either a self-replicating plasmid or an integrative plasmid.

In an embodiment, the nucleic acids of interest can be amplified from nucleic acid samples using amplification techniques. PCR can be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, and for nucleic acid sequencing.

In order to use isolated sequences in the above techniques, recombinant DNA vectors suitable for transformation of cyanobacteria can be prepared. Techniques for transformation are well known and described in the technical and scientific literature. For example, a DNA sequence encoding one or more of the genes described herein can be combined with transcriptional and other regulatory sequences which will direct the transcription of the sequence from the gene in the transformed cyanobacteria.

In an embodiment, an antibiotic resistance cassette for selection of positive clones can be present on the plasmid to aid in selection of transformed cells. For example, genes conferring resistance to ampicillin, gentamycin, kanamycin, or other antibiotics can be inserted into the vector, under the control of a suitable promoter. Other antibiotic resistance genes can be used if desired. In some embodiments, the vector contains more than one antibiotic resistance gene. The presence of a foreign gene encoding antibiotic resistance can be selected, for example, by placing the putative transformed cells into a suitable amount of the corresponding antibiotic, and picking the cells that survive.

In an embodiment, the genes of interest are inserted into the cyanobacterial chromosome. When the cell is polyploid, the gene insertions can be present in all of the copies of the chromosome, or in some of the copies of the chromosome.

In another embodiment, the inserted genes are present on an extrachromosomal plasmid. The extrachromosomal plasmids can be present in a high number or a low number within the genetically enhanced cyanobacterium.

The extrachromosomal plasmid can be derived from an outside source, such as, for example, RSF1010-based plasmid vectors, or it can be derived from an endogenous plasmid from the cyanobacterial cell or from another species of cyanobacteria.

Many cyanobacterial species harbor endogenous vectors that can be used to carry production genes. The cyanobacterium Synechococcus PCC 7002, for example, contains six endogenous plasmids having different numbers of copies in the cyanobacterial cell (Xu et al., 2011, “Expression of genes in cyanobacteria: Adaption of Endogenous Plasmids as platforms for High-Level gene Expression in Synechococcus PCC 7002”, Photosynthesis Research Protocols, Methods in Molecular Biology, 684:273-293). The endogenous plasmid pAQ1 is present in a number of 50 copies per cell (high-copy), the plasmid pAQ3 with 27 copies, the plasmid pAQ4 with 15 copies and the plasmid pAQ5 with 10 copies per cell (low-copy). In an embodiment, these endogenous plasmids can be used as an integration platform for the 1,2-propanediol genes described herein. The propanediol pathway genes can be integrated into the endogenous cyanobacterial plasmids via homologous recombination, or by other suitable means. It is also possible to create a “shuttle vector” based on the backbone of an endogenous vector, in combination with portions of self-replicating E. coli vectors, for ease of genetic manipulation. Such vectors can be easily manipulated in E. coli, for example, then the vectors can be transferred to the cyanobacterial host strain for the production of 1,2-propanediol.

In an embodiment, the inserted genes are present on an extrachromosomal plasmid, wherein the plasmid has multiple copies per cell. The plasmid can be present, for example, at about 1, 3, 5, 8, 10, 15, 20, 30, 40, 50, 60, 70, 80, 90, or more copies per host cyanobacterial cell. In an embodiment, the plasmids are fully segregated.

In another embodiment, the inserted genes are present on one cassette driven by one promoter. In another embodiment, the inserted genes are present on separate plasmids, or on different cassettes.

In another embodiment, the inserted genes are modified for optimal expression by modifying the nucleic acid sequence to accommodate the cyanobacterial cell's protein translation system. Modifying the nucleic acid sequences in this manner can result in an increased expression of the genes.

The inserted genes can be regulated by one promoter, or they can be regulated by individual promoters. The promoters can be constitutive or inducible. The promoter sequences can be derived, for example, from the host cell, from another organism, or can be synthetically derived.

Any desired promoter can be used to regulate the expression of the genes for 1,2-propanediol production. Exemplary promoter types include but are not limited to, for example, constitutive promoters, inducible promoters (e.g., by nutrient starvation, heat shock, mechanical stress, environmental stress, metal concentration, light exposure, etc.), endogenous promoters, heterologous promoters, and the like.

In an embodiment, the inserted genes for 1,2-propanediol production are placed under the transcriptional control of promoters selected from a group consisting of: rbcL, ntcA, nblA, isiA, petJ, petE, sigB, lrtA, htpG, hspA, clpB1, hliB, ggpS, psbA2, psaA, nirA, crhC, and srp. The promoters hspA, clpB1, and hliB can be induced by heat shock (raising the growth temperature of the host cell culture from 30° C. to 40° C.), cold shock (reducing the growth temperature of the cell culture from 30° C. to 20° C.), oxidative stress (for example by adding oxidants such as hydrogen peroxide to the culture), or osmotic stress (for example by increasing the salinity). The promoter sigB can be induced by stationary growth, heat shock, and osmotic stress. The promoters ntcA and nblA can be induced by decreasing the concentration of nitrogen in the growth medium and the promoters psaA and psbA2 can be induced by low light or high light conditions. The promoter htpG can be induced by osmotic stress and heat shock. The promoter crhC can be induced by cold shock. An increase in copper concentration can be used in order to induce the promoter petE, whereas the promoter petJ is induced by decreasing the copper concentration. The promoter srp can be induced by the addition of IPTG (isopropyl β-D-1-thiogalactopyranoside). Additional details of these promoters can be found, for example, in PCT/EP2009/060526, which is incorporated by reference herein in its entirety.

In an embodiment, the inducible promoters are selected from the group consisting of: PntcA, PnblA, PisiA, PpetJ, PpetE, PggpS, PpsbA2, PpsaA, PsigB, PlrtA, PhtpG, PnirA, PhspA, PclpB1, PhliB, PcrhC, PziaA, PsmtA, PcorT, PnrsB, PaztA, PbmtA, Pbxal, PzntA, PczrB, PnmtA and Psrp.

In certain other embodiments, truncated or partially truncated versions of these promoters including only a small portion of the native promoters upstream of the transcription start point, such as the region ranging from −35 to the transcription start can often be used. Furthermore, the introduction of nucleotide changes into the promoter sequence, e.g. into the TATA box, the operator sequence and/or the ribosomal binding site (RBS) can be used to tailor or optimize the promoter strength and/or its induction conditions, such as the concentration of inducer compound.

In an embodiment, the promoter used to regulate expression of 1,2-propanediol pathway genes is the Psrp promoter (SEQ ID NO: 1). In another embodiment, the promoter is PnblA₇₁₂₀ (the phycobilisome degradation protein promoter from Nostoc sp. PCC 7120 (SEQ ID NO: 2).

In an embodiment, the promoter is PrbcL₆₈₀₃ (the constitutive ribulose 1,5-bisphosphate carboxylase/oxygenase large subunit promoter from Synechocystis sp. PCC 6803 (SEQ ID NO: 3). The promoter can also be a derivative or a variant of an rbcL promoter, such as the PrbcL promoter from Synechocystis sp. PCC 6803, or from another organism. Thus, in an embodiment, the promoter is Prbc-_{PCC 6803} (SEQ ID NO: 4). In another embodiment, the promoter is PrbcL*-_{PCC 6803} (SEQ ID NO: 5). In another embodiment, the promoter is Prbe*-_{PCC 6803} (SEQ ID NO: 6).

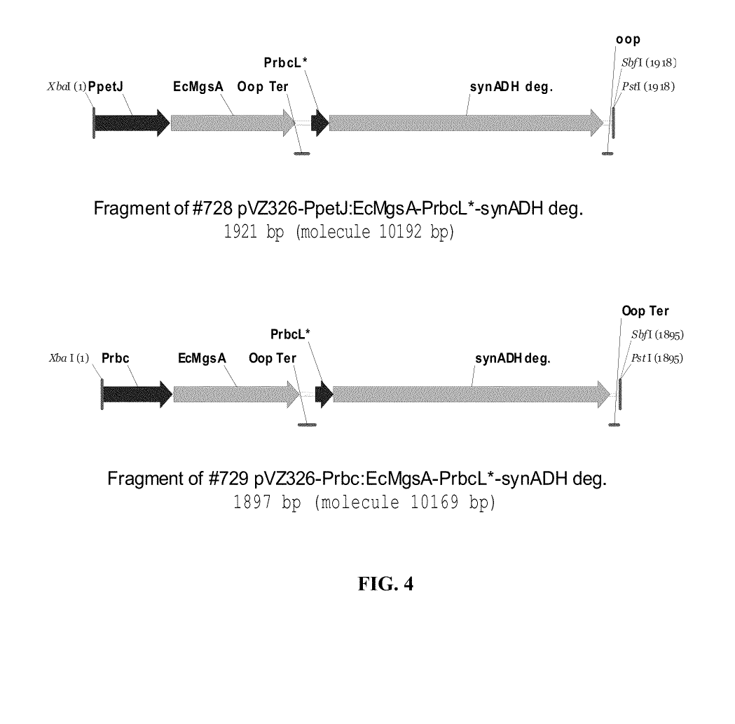

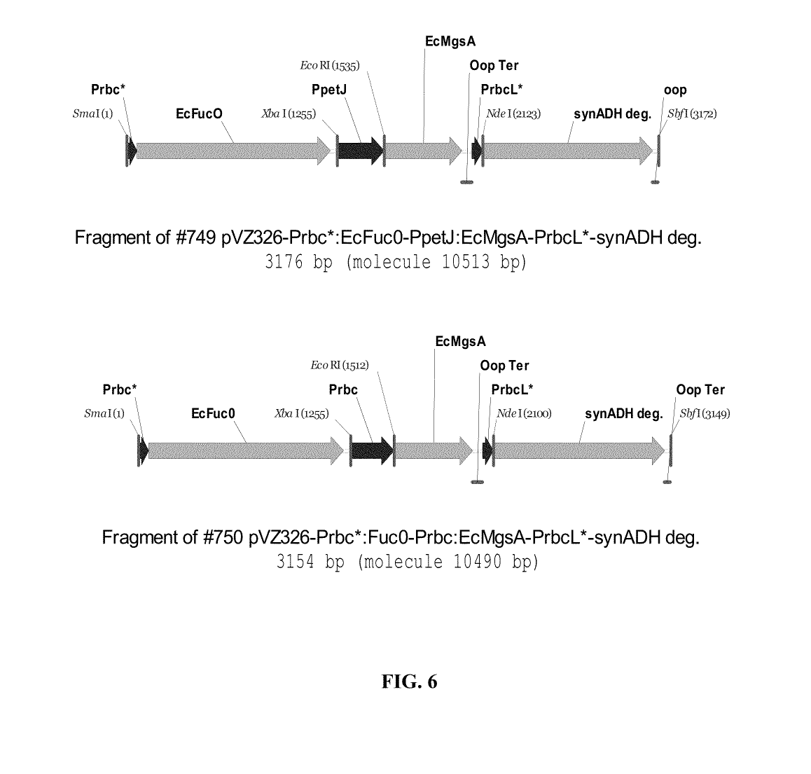

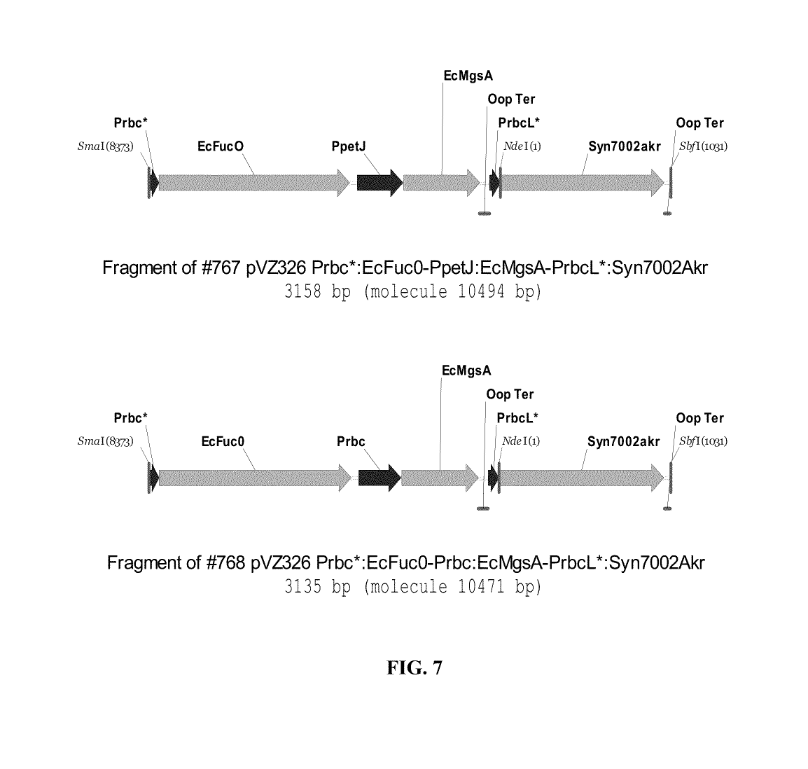

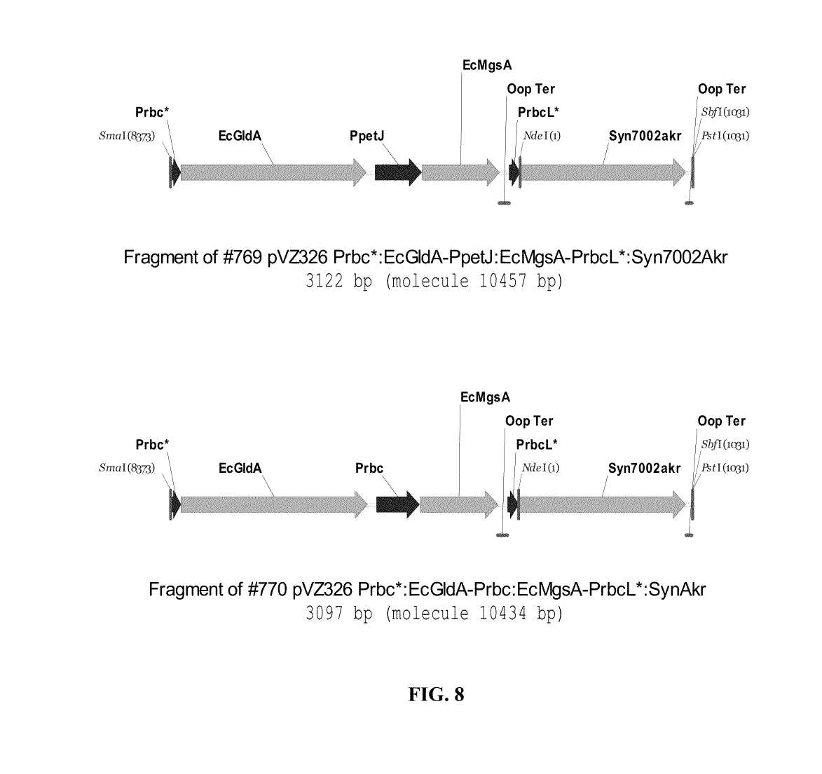

Examples of other promoters that can be used are PsmtA₇₀₀₂ (the promoter for prokaryotic metallothionein-related protein from Synechococcus sp. PCC 7002; (SEQ ID NO: 7); and the repressor/promoter system ziaR-PziaA₆₈₀₃ (the zinc-inducible promoter from Synechocystis sp. PCC 6803; (SEQ ID NO: 8). Additionally, the promoter PpetJ from Synechocystis sp. PCC 6803 (SEQ ID NO: 9) can also be used, as shown in FIG. 4 through FIG. 8.

A terminator region can be inserted at the 3′ end of the genes of interest. An exemplary terminator sequence is the Oop lamda phage terminator (SEQ ID NO: 10), as shown in the gene cassette maps shown in FIG. 4 through FIG. 7.

### 1,2-Propanediol

1,2-propanediol (also termed propylene glycol, propane-1,2-diol, 1,2-dihydroxypropane, and methylethylene glycol) is a three-carbon diol with a stereogenic center at the central carbon atom. The enantiomerically pure 1,2-propanediol generated from biological processes is a high value commodity chemical with a broad application as solvent, food additive, de-icing compounds etc.

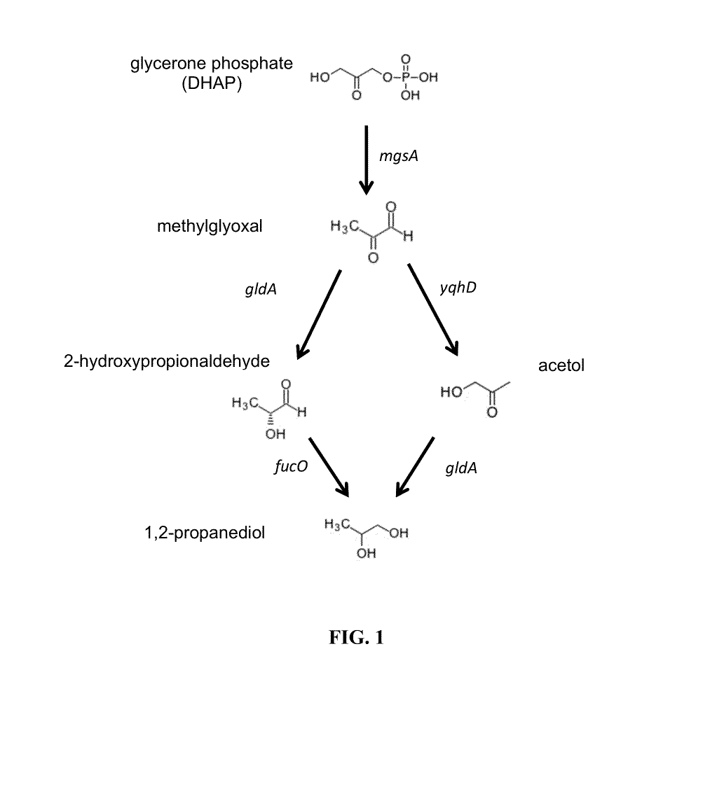

In an embodiment, the biochemical pathway from CO₂ to 1,2-propanediol involves several steps, as shown in FIG. 1. Briefly, these steps are:

CO₂→→→Dihydroxyacetone phosphate(DHAP)→Methylglyoxal→Lactaldehyde→1,2-Propanediol

or

CO₂→→→Dihydroxyacetone phosphate→Methylglyoxal→Aceto→1,2-Propanediol

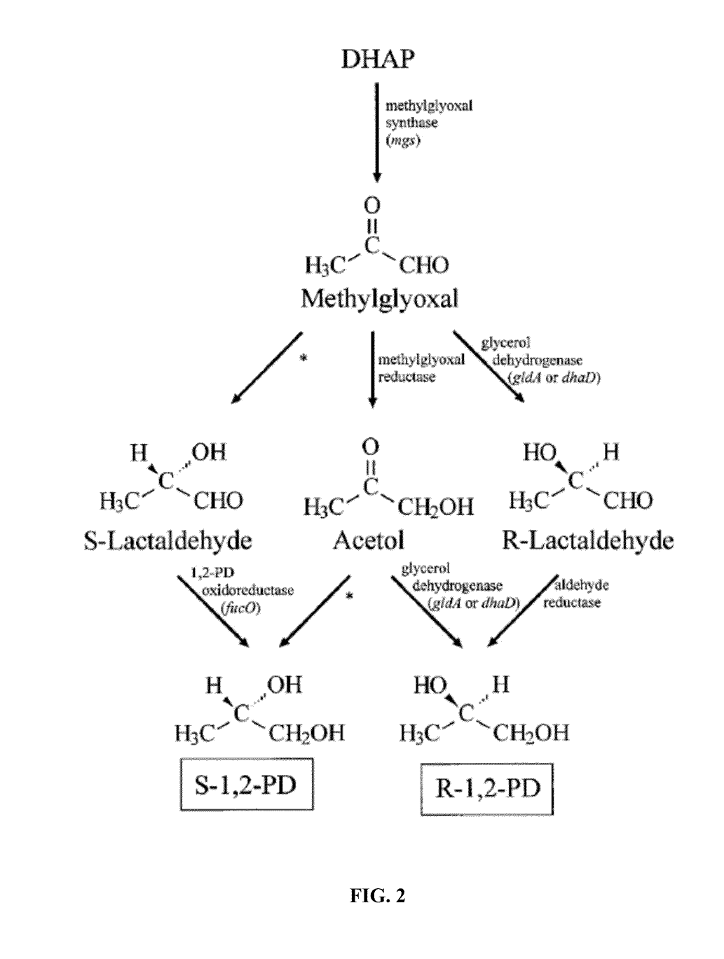

FIG. 2 shows another diagram of 1,2-propanediol production in E. coli (Alteras et al., 1999, Applied and Envir. Biol. 65:1180-1185). The diagram shows biosynthetic pathway variations that can be used to produce either S-1,2-propanediol or R-1,2-propanediol in E. coli cells.

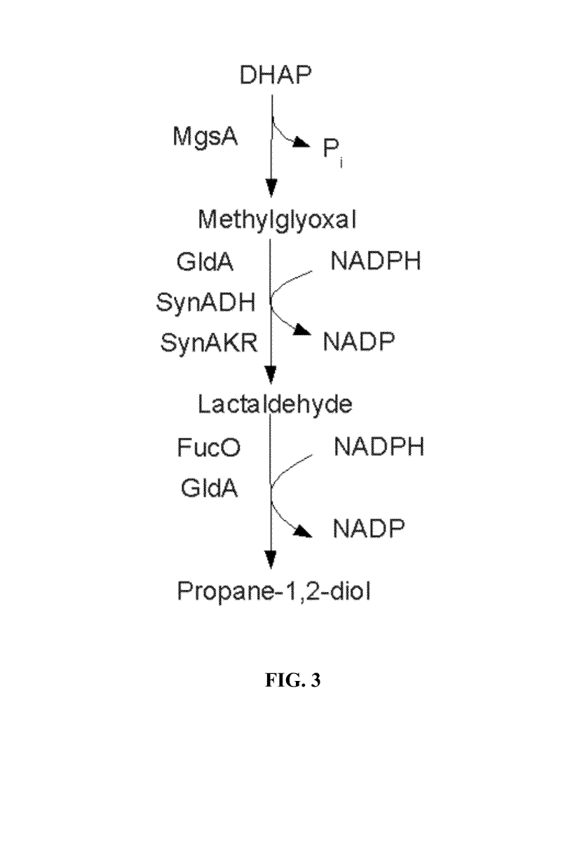

Yet another biosynthetic pathway diagram is shown in FIG. 3, where possible alternate enzymes for some of the steps are indicated. Examples 3 through 6 demonstrate how the gene cassettes encoding the various enzymes were transferred to cyanobacterial cells, then tested for activity in cyanobacteria.

### Production of 1,2-Propanediol in Cyanobacteria

Cyanobacteria can be modified to produce 1,2-propanediol. In an embodiment of the invention, in order to create the 1,2-propanediol biosynthetic pathway from CO₂ as the carbon source, the following genes can be inserted into the cyanobacterial cell:
gldA-fucO-mgsA (“GFM”)

In another embodiment of the invention, the gene yqhD can also be inserted into the cyanobacterial cell. As shown in FIG. 1, the biosynthetic pathway for 1,2-propanediol production can include a branch from the intermediate methylglyoxal—both the enzymes GldA and YqhD can utilize methylglyoxal to move the pathway toward 1,2-propanediol production. Methylglyoxal is partially toxic to many cell types, so the inclusion of another enzyme and another biosynthetic pathway branch can help deplete any methylglyoxal that accumulates from the prior steps. Further, since YqhD acts on methylglyoxal to create acetol, which, in the presence of GldA, becomes 1,2-propanediol, the added branching step can also produce more 1,2-propanediol.
gldA-yqhD-fucO-mgsA (“GYFM”)

In another embodiment, the biosynthetic pathway from:

CO2→→→DHAP→Methylglyoxal→Lactaldehyde→1,2-Propanediol

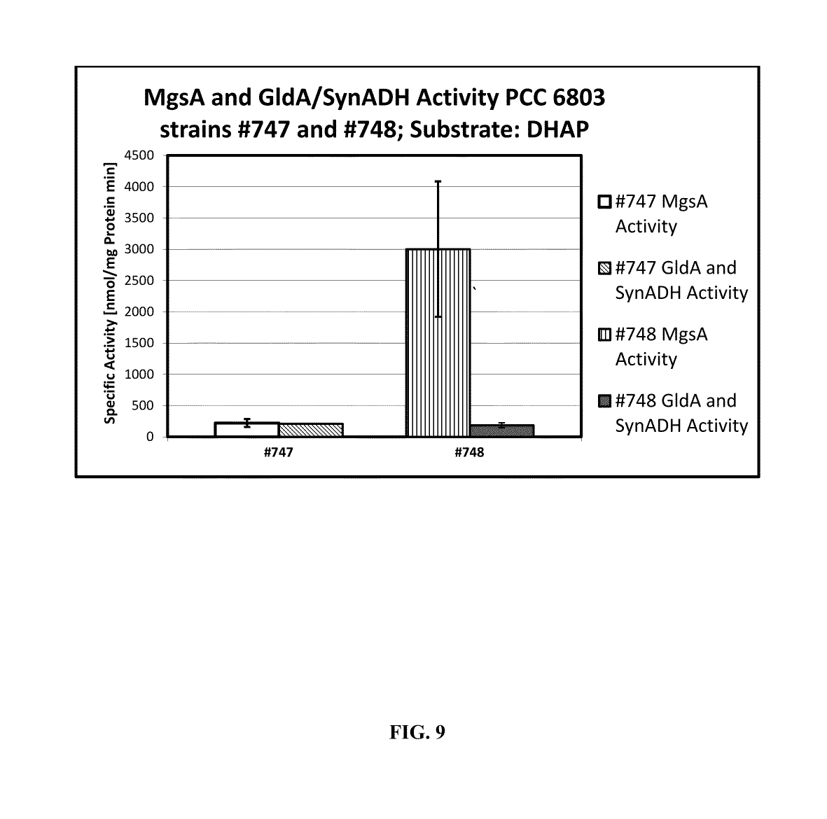

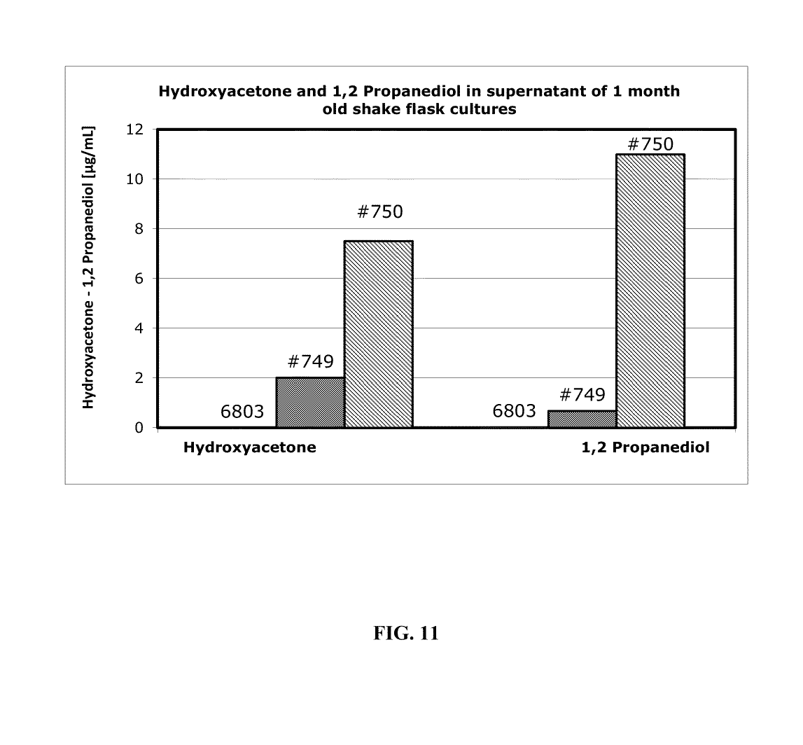

can also be achieved with a choice of several enzymes, as shown in FIG. 3. The conversion of methylglyoxal to lactaldehyde can be achieved, for example, by either GldA, ADH, or AKR. The conversion of lactaldehyde to 1,2-propanediol can be achieved, for example, by either FucO or GldA. To determine the effect of each of these enzyme choices on the production of the intermediates and the end product 1,2-propanediol, different combinations of the above-described enzymes were constructed as shown in Example 3. Plasmids containing the various gene cassettes as shown in Table 3 (such as those shown in FIGS. 4 through 8) were transferred to cyanobacterial host cells using a shuttle vector system. The enzyme activity of the resulting transformed cultures was confirmed as shown in Table 3. In certain cases, such as for the #748 and #749 constructs, the intermediates and the end product 1,2-propanediol were also tested and confirmed. When transferred to a cyanobacterial cell, the enzyme activities and levels of intermediates could be determined, as demonstrated in FIGS. 9 through 14.

A demonstration of the construction of plasmids for the production of 1,2-propanediol is shown in Examples 3 and 10. An example of a successful transformation to cyanobacteria is shown in Example 12. Verification of the successful transformation is shown in Example 13. A suitable method for determining the level of 1,2-propanediol that is produced is shown in Example 14.

The terms “gldA” and “glycerol dehydrogenase” refer to an enzyme that facilitates the formation of 1,2-propanediol from acetol, or, alternatively, the formation of 2-hydroxypropionaldehyde (lactaldehyde) from methylglyoxal. A “gldA gene” is a nucleic acid that encodes the enzyme. In an embodiment, the gene is originally derived from E. coli. In an embodiment, the gene is nucleic acid sequence Accession No. NC_{—}010473.1; encoding a protein having Accession No. YP_{—}001732735.1. In another embodiment, the invention provides a recombinant photosynthetic microorganism that includes at least one heterologous DNA sequence encoding at least one polypeptide that catalyzes a substrate to product conversion that leads to the synthesis of 1,2-propanediol from acetol, or, alternatively, the formation of 2-hydroxypropionaldehyde from methylglyoxal. In an embodiment, the GldA enzyme is in the enzyme class EC#1.1.1.6. In an embodiment, the GldA nucleotide sequence is SEQ ID NO: 11 and the amino acid sequence is SEQ ID NO: 12.

The terms “FucO” and “L-1,2-propanediol oxidoreductase” refer to an enzyme that facilitates the formation of 1,2-propanediol from 2-hydroxypropionaldehyde (lactaldehyde), as shown in FIG. 1. A “fucO gene” refers to the gene encoding the enzyme. In an embodiment, the gene is originally derived from E. coli. In another embodiment, the gene is nucleic acid sequence Accession No. NC_{—}010473.1; encoding a protein having accession #YP_{—}001731690.1. In another embodiment, the invention provides a recombinant photosynthetic microorganism that includes at least one heterologous DNA sequence encoding at least one polypeptide that catalyzes a substrate to product conversion that leads to the synthesis of 1,2-propanediol from 2-hydroxypropionaldehyde (lactaldehyde). In an embodiment, the FucO enzyme is in the enzyme class EC#1.1.1.77. In another embodiment, the FucO enzyme is present in the broader enzyme class EC#1.1.1.21. In an embodiment, the FucO nucleotide sequence is SEQ ID NO: 13, and the FucO amino acid sequence is SEQ ID NO: 14.

The terms “MgsA” and “methylglyoxal synthase” refer to an enzyme that facilitates the formation of methylglyoxal from glycerone phosphate. An “mgsA gene” refers to the gene encoding the enzyme. Details of the regulation of this enzyme in E. coli, as well as an assay for its activity, can be found in Hopper et al., (1971), FEBS Letters 13:213-216. In an embodiment, the gene is originally derived from E. coli. In another embodiment, the gene sequence is nucleic acid sequence Accession No. NC_{—}010473.1; encoding a protein having accession #YP_{—}001729941.1. In another embodiment, the invention provides a recombinant photosynthetic microorganism that includes at least one heterologous DNA sequence encoding at least one polypeptide that catalyzes a substrate to product conversion that leads to the synthesis of methylglyoxal from glycerone phosphate. In an embodiment, the MgsA enzyme is a member of the enzyme class EC#4.2.3.3. In an embodiment, the mgsA nucleotide sequence is SEQ ID NO: 15, and the mgsA amino acid sequence is SEQ ID NO: 16.

The term “yqhD” refers to a gene encoding an alcohol dehydrogenase. The enzyme can be utilized to form acetol from methylglyoxal (FIG. 1). In an embodiment, the gene is derived from E. coli. In an additional embodiment, the gene is nucleic acid accession #NC_{—}010473.1:3251122.3252285 and the protein accession is #YP_{—}001731875.1. In an embodiment, the YqhD nucleotide sequence is SEQ ID NO: 17, and the YqhD amino acid sequence is SEQ ID NO: 18.

The term “AKR” or “sacR1” refers to a gene encoding the enzyme aldo/keto reductase. In an embodiment, the gene is from a cyanobacterial species, such as Synechococcus. In an embodiment, the gene is Cyanobase ID #SYNPCC7002_A1474. In an embodiment, the AKR sequence is synAKR, and its nucleotide sequence is SEQ ID NO: 19, while the synAKR amino acid sequence is SEQ ID NO: 20.

The term “synADH” or “ADH” refers to a gene encoding the enzyme alcohol dehydrogenase. As shown in FIG. 3, it is possible that this enzyme can catalyze the conversion of methylglyoxal to lactaldehyde, which is a portion of the biosynthetic pathway leading to the production of 1,2-propanediol. In an embodiment, the synADH gene is a codon optimized version of the gene originally derived from Synechocystis PCC 6803 (nucleic acid SEQ ID NO: 21, amino acid SEQ ID NO: 22). In another embodiment, ADH from another source can be used.

In certain cyanobacterial strains that are capable of metabolizing glycerol, the input carbon source can be glycerol in addition to, or instead of CO₂. The cyanobacterial strain Synechococcus sp. PCC 7002, for example, naturally contains the genes capable of glycerol metabolism. Thus, this strain is a good candidate for using a glycerol feed to produce 1,2-propanediol.

The invention also comprises recombinant nucleic acids having 80%, 85%, 90%, 95%, 97%, 98%, 99% identity to SEQ ID NOs: 11, 13, 15, 17, 19 or 21.

### Transformation of Cyanobacterial Cells

Cyanobacteria can be transformed by several suitable methods. Exemplary cyanobacteria that can be transformed with the nucleic acids described herein include, but are not limited to, Synechocystis, Synechococcus, Acaryochloris, Anabaena, Thermosynechococcus, Chamaesiphon, Chroococcus, Cyanobacterium, Cyanobium, Dactylococcopsis, Gloeobacter, Gloeocapsa, Gloeothece, Microcystis, Prochlorococcus, Prochloron, Chroococcidiopsis, Cyanocystis, Dermocarpella, Myxosarcina, Pleurocapsa, Stanieria, Xenococcus, Arthrospira, Borzia, Crinalium, Geitlerinema, Halospirulina, Leptolyngbya, Limnothrix, Lyngbya, Microcoleus, Cyanodictyon, Aphanocapsa, Oscillatoria, Planktothrix, Prochlorothrix, Pseudanabaena, Spirulina, Starria, Symploca, Trichodesmium, Tychonema, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, Iyengariella, Stigonema, Rivularia, Scytonema, Tolypothrix, Cyanothece, Phormidium, Adrianema, and the like.

Exemplary methods suitable for transformation of Cyanobacteria, include, as nonlimiting examples, natural DNA uptake (Chung, et al. (1998) FEMS Microbiol. Lett. 164: 353-361; Frigaard, et al. (2004) Methods Mol. Biol. 274: 325-40; Zang, et al. (2007) J. Microbiol. 45: 241-245), conjugation, transduction, glass bead transformation (Kindle, et al. (1989) J. Cell Biol. 109: 2589-601; Feng, et al. (2009) Mol. Biol. Rep. 36: 1433-9; U.S. Pat. No. 5,661,017), silicon carbide whisker transformation (Dunahay, et al. (1997) Methods Mol. Biol. (1997) 62: 503-9), biolistics (Dawson, et al. (1997) Curr. Microbiol. 35: 356-62; Hallmann, et al. (1997) Proc. Natl. Acad. USA 94: 7469-7474; Jakobiak, et al. (2004) Protist 155:381-93; Tan, et al. (2005) J. Microbiol. 43: 361-365; Steinbrenner, et al. (2006) Appl Environ. Microbiol. 72: 7477-7484; Kroth (2007) Methods Mol. Biol. 390: 257-267; U.S. Pat. No. 5,661,017) electroporation (Kjaerulff, et al. (1994) Photosynth. Res. 41: 277-283; Iwai, et al. (2004) Plant Cell Physiol. 45: 171-5; Ravindran, et al. (2006) J. Microbiol. Methods 66: 174-6; Sun, et al. (2006) Gene 377: 140-149; Wang, et al. (2007) Appl. Microbiol. Biotechnol. 76: 651-657; Chaurasia, et al. (2008) J. Microbiol. Methods 73: 133-141; Ludwig, et al. (2008) Appl. Microbiol. Biotechnol. 78: 729-35), laser-mediated transformation, or incubation with DNA in the presence of or after pre-treatment with any of poly(amidoamine) dendrimers (Pasupathy, et al. (2008) Biotechnol. J. 3: 1078-82), polyethylene glycol (Ohnuma, et al. (2008) Plant Cell Physiol. 49: 117-120), cationic lipids (Muradawa, et al. (2008) J. Biosci. Bioeng. 105: 77-80), dextran, calcium phosphate, or calcium chloride (Mendez-Alvarez, et al. (1994) J. Bacteriol. 176: 7395-7397), optionally after treatment of the cells with cell wall-degrading enzymes (Perrone, et al. (1998) Mol. Biol. Cell 9: 3351-3365); and biolistic methods (see, for example, Ramesh, et al. (2004) Methods Mol. Biol. 274: 355-307; Doestch, et al. (2001) Curr. Genet. 39: 49-60; all of which are incorporated herein by reference in their entireties).

### Culturing the Cyanobacterial Cells

In an embodiment, 1,2-propanediol is synthesized in cyanobacterial cultures by preparing host cyanobacterial cells having the gene constructs discussed herein, and then growing cultures of the cells.

The choice of culture medium can depend on the cyanobacterial species. In an embodiment of the invention, the following BG-11 medium for growing cyanobacteria can be used (Table 1 and Table 2, below). When salt water species are grown, Instant Ocean (35 g/L) and vitamin B₁₂ (1 μg/ml) can be added to the culture medium.

TABLE 1
Exemplary Culture Medium Composition

| | | |
|---|---|---|
| | Amount (per | Final |
| Compound | liter) | Concentration |

| NaNO₃ | 1.5 | g | 17.6 | mM |
|---|---|---|---|---|
| K₂HPO₄ | 0.04 | g | 0.23 | mM |
| MgSO₄•7H₂O | 0.75 | g | 3.04 | mM |
| CaCl₂•2H₂O | 0.036 | g | 0.24 | mM |
| Citric acid | 0.006 | g | 0.031 | mM |
| Ferric ammonium citrate | 0.006 | g | — | |
| EDTA (disodium salt) | 0.001 | g | 0.0030 | mM |
| NaCO₃ | 0.02 | g | 0.19 | mM |

| | | | |
|---|---|---|---|
| Trace metal mix A5 | 1.0 | ml | — |

TABLE 2
Trace Metal Mix

| | | | Concentration in | |
|---|---|---|---|---|
| Trace Metal mix A5 | | | Final Medium | |
| H₃BO₃ | 2.86 | g | 46.26 | μM |
| MnCl₂•4H₂O | 1.81 | g | 9.15 | μM |
| ZnSO₄•7H₂O | 0.222 | g | 0.772 | μM |
| NaMoO₄•2H₂O | 0.39 | g | 1.61 | μM |
| CuSO₄•5H₂O | 0.079 | g | 0.32 | μM |
| Co(NO₃)₂•6H₂O | 49.4 | mg | 0.170 | μM |

| | | | |
|---|---|---|---|
| Distilled water | 1.0 | L | — |

In an embodiment, the cells are grown autotrophically, and the only carbon source is CO₂. In another embodiment, the cells are grown mixotrophically, for example with the addition of a carbon source such as glycerol.

The cultures can be grown indoors or outdoors. The cultures can be axenic or non-axenic. In another embodiment, the cultures are grown indoors, with continuous light, in a sterile environment. In another embodiment, the cultures are grown outdoors in an open pond type of photobioreactor.

In an embodiment, the cyanobacteria are grown in enclosed bioreactors in quantities of at least about 100 liters, 500 liters, 1000 liters, 2000 liters, 5,000 liters, or more. In an embodiment, the cyanobacterial cell cultures are grown in disposable, flexible, tubular photobioreactors made of a clear plastic material.

The light cycle can be set as desired, for example: continuous light, or 16 hours on and 8 hours off, or 14 hours on and 10 hours off, or 12 hours on and 12 hours off.

### Confirmation of Transformation and Enzyme Activity in the Transformed Cyanobacterial Cells

The presence of the 1,2-propanediol pathway genes in the shuttle vector or in the transformed host cell can be determined by several means. In an embodiment, the gene pathway cassette is confirmed using PCR-based methods. The presence of the expressed mRNA can be determined, for example, using RT-PCR or a northern blot, or by any other suitable means. The presence of the expressed enzymes themselves can be determined, for example, by an SDS-PAGE followed by transfer to a western blot. In order to confirm that the genes encoding the 1,2-propanediol pathway enzymes are actually functional when expressed in cyanobacteria, chemical analysis, such as gas chromatography, can be performed. Specific enzymatic assays can also be performed. Examples of several enzyme activity assays for mgsA, synAKR, and FucO are described in Examples 6 through 9.

Isolation and Purification of 1,2-Propanediol from the Cyanobacterial Cultures

Various methods can be used to remove 1,2-propanediol from the cyanobacterial culture medium. In an embodiment, the propanediol is separated from the culture medium periodically as the culture is growing. For example, the culture medium can be separated from the cells, followed by a filtration step. The propanediol can then be removed from the filtrate. The culture medium can be recycled back into the culture, if desired, or new culture medium can be added. In another embodiment, the propanediol is removed from the culture at the end of the batch run.

Another method of separating polyol products from the culture producing it is described in International Patent Application No. WO/2000/024918 to Fisher et al. This application describes a pre-treatment step that can be used to separate the cells from the polyol-containing solution without killing the cell culture. Additional steps can include flotation or flocculation to remove proteinaceous materials, followed by ion exchange chromatography, activated carbon treatment, evaporative concentration, precipitation and crystallization.

A process for reclaiming 1,2-propylene glycol from operative fluids such as antifreeze solutions, heat transfer fluids, deicers, lubricants, hydraulic fluids, quenchants, solvents and absorbents, is disclosed in U.S. Pat. No. 5,194,159 to George et al. The method involves contacting the fluid with semi-permeable membranes under reverse osmosis.

U.S. Pat. No. 5,510,036 to Woyciesjes et al. discloses a process for the purification and removal of contaminants (such as heavy metals oils and organic contaminants) in a polyol-containing solution, wherein the process involves lowering the pH and adding precipitating, flocculating, or coagulating agents, which can be followed by filtration and an ion exchange chromatography step.

The present invention is further described by the following non-limiting examples. However, it will be appreciated that those skilled in the art, on consideration of this disclosure, may make modifications and improvements within the spirit and scope of the present invention.

### EXAMPLES

### Example 1

### General Methods

Restriction endonucleases were purchased from New England Biolabs (New England Biolabs (NEB), Ipswich, Mass.), unless otherwise noted. PCR was performed using an Eppendorf Mastercycler thermocycler (Eppendorf, Hauppauge, N.Y.), using Phire II Hot Start polymerase or Taq DNA polymerase (NEB) for diagnostic amplifications, and Phusion polymerase or Crimson LongAmp Taq Polymerase (NEB) for high fidelity amplifications. PCR temperature profiles were set up as recommended by the polymerase manufacturer. Cloning was performed in E. coli using XL10-Gold Ultracompetent cells (Agilent Technologies, Santa Clara, Calif.) following the manufacturer's protocol. TOPO cloning kits (Zero Blunt TOPO PCR Cloning kit) were purchased from Invitrogen (Invitrogen, Carlsbad, Calif.), and were used according to the manufacturer's protocol.

BG-11 stock solution was purchased from Sigma Aldrich (Sigma Aldrich, St. Louis, Mo.). Marine BG-11 (MBG-11) was prepared by dissolving 35 g Instant Ocean (United Pet Group, Inc, Cincinnati, Ohio) in 1 L water and supplementing with BG-11 stock solution. Vitamin B₁₂ (Sigma Aldrich) was supplemented to MBG-11 to achieve a final concentration of 1 μg/L, as needed. Solid media (agar plates) were prepared similarly to liquid media, with the addition of 1% (w/v) phyto agar (Research Products International Corp, Mt. Prospect, Ill.). Stock solutions of the antibiotics spectinomycin (100 mg/ml) and kanamycin (50 mg/ml) were purchased from Teknova (Teknova, Hollister, Calif.). Stock solution of the antibiotic gentamycin (10 mg/ml) was purchased from MP Biomedicals (MP Biomedicals, Solon, Ohio).

### Example 2

### SLIC Method (Sequence- and Ligation-Independent Cloning)

Primers were designed with 5′ sequences that overlapped the target vector at the desired restriction site, or which overlapped the next PCR product if inserting more than one product at a time. The overlapping sequence was typically 30 base pairs (bp) long. PCR products were amplified from genomic DNA (Klebsiella or Saccharomyces) or from whole cells (E. coli) and gel-purified. Target vectors were digested with appropriate restriction enzymes and gel-purified. To generate the 30-bp sticky ends, digested target vector (200 ng-1 μg) and each PCR product (20 ng-1 μg) were treated with 0.5 U of T4 DNA polymerase from NEB in NEB buffer 2 plus BSA (with no dNTP's) and incubated at room temperature for 15 minutes per 10 bp overlap (45 minutes for a 30 bp overlap). Reactions were stopped by adding 1/10 volume of 10 mM dCTP (or other single dNTP). Equimolar amounts (1:1 or 1:1:1, etc.) of T4-treated vector and insert(s) were combined in 8 μl volume in a PCR tube. 10× T4 ligase buffer, 1 μl, was added to the tube. Using a thermal cycler, reactions were heated to 65° C. for 10 minutes, then slowly ramped down to 37° C. (10% ramp speed). RecA protein from NEB, 20 ng in 1 ml 10× RecA buffer, was added to the tube, which was incubated at 37° C. for 30 minutes. 5 μA of the reaction was used for E. coli transformation.

### Example 3

### Construction of Various Gene Cassettes for Confirmation of Activity of Certain Pathway Enzymes for 1,2-Propanediol Production

FIG. 3 shows a biosynthetic pathway scheme for production of 1,2-propanediol in Cyanobacteria. To confirm whether each of the putative pathway enzymes would actually be correctly expressed and have normal activity in a cyanobacterial species, various constructs were prepared as shown below in Table 3. Maps of the gene constructs are shown in FIG. 4 through FIG. 8. The constructs varied in the choice of promoter, the choice of antibiotic resistance gene used, and the choice of enzyme.

TABLE 3
Gene Cassettes for Portions of 1,2-Propanediol Pathway

| | | | Antibiotic | | | | | |
|---|---|---|---|---|---|---|---|---|
| | | | Resistance | Primer | Restriction | Restriction | | Enzyme |
| Plasmid # | Plasmid Name | Promoter | Gene | used | Sites | Sites | Comments | Assays |
| 550 | pVZ325-PpetJ- | petJ, rbcL* | Gm/Spec | / | SpeI/PstI | SpeI/PstI | Recipients; | |
| | PDC-PrbcL*- | | | | | | Plasmid SEQ ID NO: | |
| | synADH deg. | | | | | | 23 | |
| 707 | pJet-PpetJ-fbpI | petJ | Amp | / | XbaI/EcoRI | XbaI/EcoRI | recipient for EcMgsA | |
| | | | | | | | 712 | |
| | | | | | | | Plasmid SEQ ID NO: | |
| | | | | | | | 24 | |
| 713 | pJet-Prbc-fbpI | rbc | Amp | / | EcoRI/XbaI | EcoRI/XbaI | recipient for EcMgsA | |
| | | | | | | | 712 | |
| | | | | | | | Plasmid SEQ ID NO: | |
| | | | | | | | 26 | |
| 712 | pJet-EcMgsA | | Amp | 313 | blunt end cloning | | E. coli MgsA aimed for | |
| | | | | and | | | cloning into #550, | |
| | | | | 314 | | | #707 and #713 | |
| | | | | | | | Plasmid SEQ ID NO: | |
| | | | | | | | 25 | |
| 717 | pJet-PpetJ:EcMgsA | petJ | Amp | / | EcoRI/XhoI | EcoRI/XhoI | PpetJ:EcMgsA aimed | |
| | | | | | | | for cloning into #550 | |
| | | | | | | | Plasmid SEQ ID NO: | |
| | | | | | | | 27 | |
| 718 | pJet-Prbc:EcMgsA | rbc | Amp | / | EcoRI/XhoI | EcoRI/XhoI | Prbc:EcMgsA aimed | |
| | | | | | | | for cloning into #550 | |
| | | | | | | | Plasmid SEQ ID NO: | |
| | | | | | | | 28 | |
| 728 | pVZ326- | petJ, rbcL | Gm | / | XbaI/SalI | XbaI/XhoI | PpetJ:EcMgsA out of | MgsA+ |
| | PpetJ:EcMgsA | | | | | | #717 and cloned into | SynADH+ |
| | PrbcL*:SynADH | | | | | | #550 | |
| | deg | | | | | | Cassette SEQ ID NO: | |
| | | | | | | | 46 | |
| 729 | pVZ326- | rbc, rbcL | Gm | / | XbaI/SalI | XbaI/XhoI | PpetJ:EcMgsA out of | MgsA+ |
| | Prbc:EcMgsA | | | | | | #718 and cloned into | SynADH+ |
| | PrbcL*:SynADH | | | | | | #550 | |
| | deg | | | | | | Cassette SEQ ID NO: | |
| | | | | | | | 47 | |
| 733 | pJet Syncoccus 7002 | | Amp | 328 | blunt end cloning | | Aldo-Ketoreductase | |
| | akr:oop NdeI PstI | | | and | | | from | |
| | | | | 329 | | | Synechococcus 7002 | |
| | | | | | | | aimed for cloning into | |
| | | | | | | | 728/729 | |
| | | | | | | | Plasmid SEQ ID NO: | |
| | | | | | | | 29 | |
| 734 | pJet Prbc*:EcGldA | Prbc* | Amp | 326 | blunt end cloning | | E. coli K12 Glycerol | |
| | XmaI XbaI | | | and | | | Dehydrogenase aimed | |
| | | | | 327 | | | for cloning into | |
| | | | | | | | 728/729 | |
| | | | | | | | Plasmid SEQ ID NO: | |
| | | | | | | | 30 | |
| 735 | pJet | Prbc* | Amp | 330 | blunt end cloning | | E. coli K12 | |
| | Prbc*:EcFuc0:oop | | | and | | | Lactaldehyde | |
| | Xma-Xba | | | 331 | | | Oxidoreductase aimed | |
| | | | | | | | for cloning into | |
| | | | | | | | 728/729 | |
| | | | | | | | Plasmid SEQ ID NO: | |
| | | | | | | | 31 | |
| 747 | pVZ326- | Prbc*, PpetJ, | Gm | — | SmaI/XbaI | SmaI/XbaI | E. coli gldA cut from | MgsA+ |
| | Prbc*:EcGldA- | PrbcL* | | | | | #734, cloned into #728 | SynADH+ |
| | PpetJ:EcMgsA- | | | | | | Cassette SEQ ID NO: | GldA+ |
| | PrbcL*-synADH | | | | | | 48 | |
| | deg. | | | | | | | |
| 748 | pVZ326- | Prbc*, Prbc, | Gm | — | SmaI/XbaI | SmaI/XbaI | E. coli gldA cut from | MgsA+ |
| | Prbc*:EcGldA- | PrbcL* | | | | | #734, cloned into #729 | SynADH+ |
| | Prbc:EcMgsA- | | | | | | Cassette SEQ ID NO: | GldA+ |
| | PrbcL*-synADH | | | | | | 49 | |
| | deg. | | | | | | | |
| 749 | pVZ326- | Prbc*, PpetJ, | Gm | | SmaI/XbaI | SmaI/XbaI | E. coli fuc0 cut from | MgsA+ |
| | Prbc*:EcFuc0- | PrbcL* | | | | | #735, cloned into #728 | SynADH+ |
| | PpetJ:EcMgsA- | | | | | | Cassette SEQ ID NO: | FucO+ |
| | PrbcL*-synADH | | | | | | 50 | |
| | deg. | | | | | | | |
| 750 | pVZ326- | Prbc*, Prbc, | Gm | | SmaI/XbaI | SmaI/XbaI | E. coli fuc0 cut from | MgsA+ |
| | Prbc*:Fuc0- | PrbcL* | | | | | #735, cloned into #729 | SynADH+ |
| | Prbc:EcMgsA- | | | | | | Cassette SEQ ID NO: | FucO+ |
| | PrbcL*-synADH | | | | | | 51 | |
| | deg. | | | | | | | |
| 767 | pVZ326 | rbc*, petJ, | Gm | — | NdeI/SbfI | NdeI/PstI | Syn7002 Akr cut from | MgsA+ |
| | Prbc*:EcFuc0- | rbcL* | | | | | #733, cloned into #749 | Akr+ |
| | PpetJ:EcMgsA- | | | | | | Cassette SEQ ID NO: | FucO+ |
| | PrbcL*:Syn7002Akr | | | | | | 52 | |
| 768 | pVZ326 | rbc*, rbc, | Gm | — | NdeI/SbfI | NdeI/PstI | Syn7002 Akr cut from | MgsA+ |
| | Prbc*:Fuc0- | rbcL* | | | | | #733, cloned into #750 | Akr+ |
| | Prbc:EcMgsA- | | | | | | Cassette SEQ ID NO: | FucO+ |
| | PrbcL*:Syn7002Akr | | | | | | 53 | |
| 769 | pVZ326 | rbc*, petJ, | Gm | — | NdeI/PstI | NdeI/PstI | Syn7002 Akr cut from | MgsA+ |
| | Prbc*:EcGldA- | rbcL* | | | | | #733, cloned into #747 | Akr+ |
| | PpetJ:EcMgsA- | | | | | | Cassette SEQ ID NO: | GldA+ |
| | PrbcL*:Syn7002Akr | | | | | | 54 | |

### Example 4

### Alternate Enzymes for Conversion of Methylglyoxal to Lactaldehyde

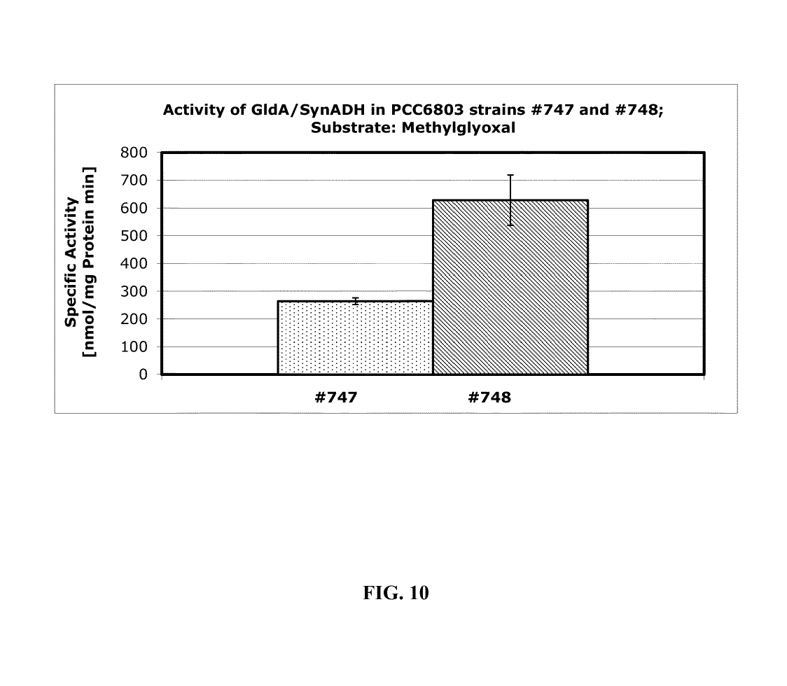

As shown in FIG. 3, it is possible that multiple enzymes (GldA, SynADH, and SynAKR) can catalyze the conversion of methylglyoxal to lactaldehyde. To confirm this, and to quantitate the products of the reaction using the different enzymes, the genes encoding these enzymes were transformed to Synechocystis sp. PCC 6803. The host cells were then measured for the presence of the gene construct and for the ability to produce the intermediate. The results show that all three enzymes GldA, SynADH and SynAKR were able to convert methylglyoxal to lactaldehyde (FIGS. 10, 13 and 14).

It was found that the enzymes GldA and SynAKR appeared to be better than SynADH for converting methylglyoxal to lactaldehyde. This may be because SynADH has a fairly low affinity to methylglyoxal (as shown in in vitro studies), causing a buildup of toxic methylglyoxal. Indeed, cultures of host cells containing the 728 and 729 plasmids, which carry the MgsA and SynADH genes, grew poorly or were lethal, which was likely to be due to the effects of accumulation of the toxic intermediate methylglyoxal in those cells.

### Example 5

### Alternate Enzymes for Conversion of Lactaldehyde to 1,2-Propanediol

As shown in FIG. 3, it is possible that multiple enzymes (FucO, GldA) can catalyze the conversion of lactaldehyde to 1,2-propanediol. To confirm this, and further to quantitate the products of the reaction using the different enzymes, the genes encoding these enzymes were transformed to Synechocystis sp. PCC 6803. The host cells were then measured for the presence of the gene construct and for the ability to produce 1,2-propanediol. The results show that both enzymes GldA and FucO were successfully expressed and their activity could be determined in vitro (FIGS. 10, 13 and 14). The activity for GldA in converting methylglyoxal to lactadehyde was higher than the rate for the conversion to 1,2-propanediol. Similar results were observed for the FucO enzymatic assay. It was concluded that the final step in this metabolic pathway is the rate limiting step for the production of 1,2-propanediol.

### Example 6

### MesA and GldA/SynADH Activity in Host Cells transformed with genes encoding GldA, MgsA, and SynADH

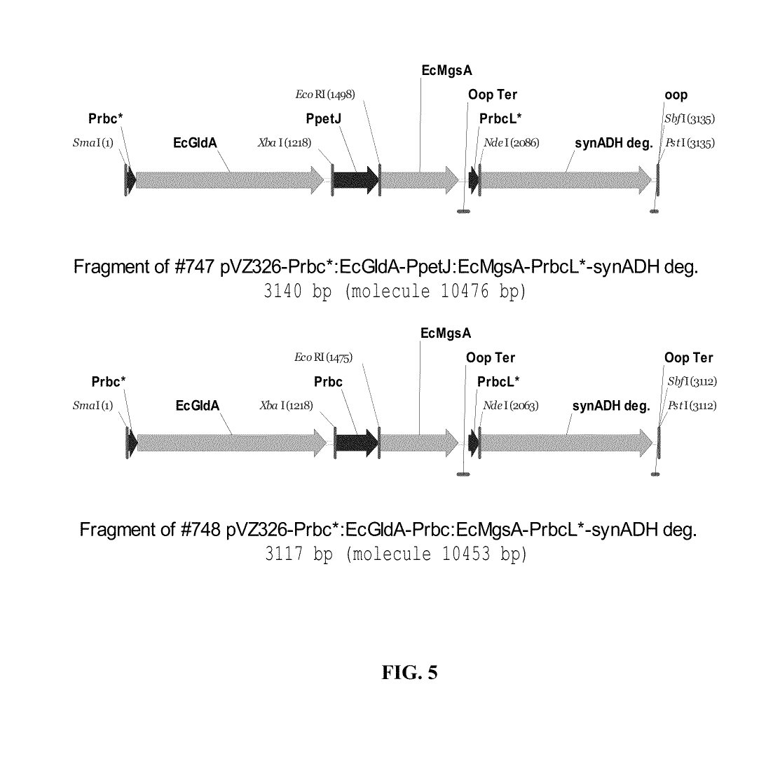

Host cyanobacterial cells were transformed with the construct #747 and #748 (FIG. 5; Table 3). Each of these constructs contained the genes encoding GldA, MgsA, and SynADH. Each of the inserted genes was controlled by its own promoter, as shown in Table 3.

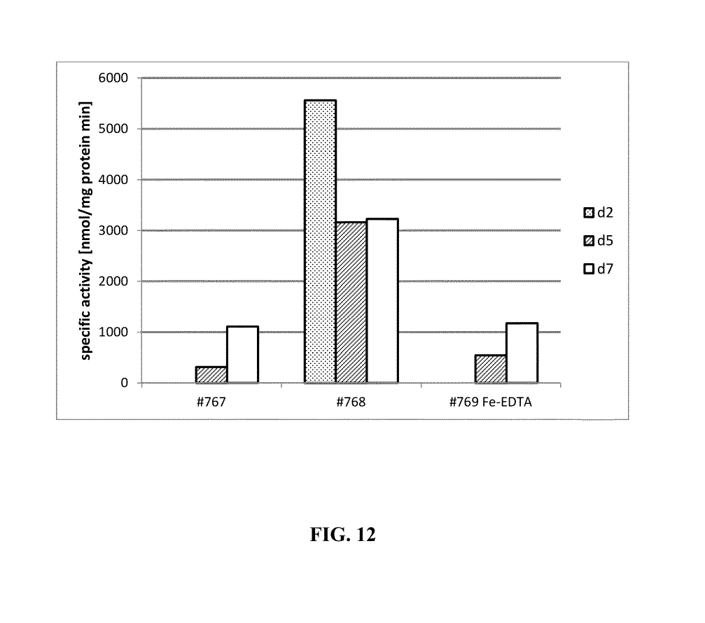

To determine MgsA activity in converting DHAP to methylglyoxal, the following method was used. Frozen pelleted cells were suspended in imidazole buffer (40 mM, pH 7) and total protein was extracted by grinding with glass beads at 30 Hz for 10 minutes. DHAP (750 μM) was added to the protein cell extract to start the reaction. Enzyme activity of MgsA was indicated by the rate of the production of methylglyoxal. Methylglyoxal reacts spontaneously with reduced glutathione, forming hemithioacetal, which is then converted to S-lactoglutathione. Thus, MgsA activity can be indirectly measured by the increase of absorption of S-lactoglutathione at OD₂₄₀ over time. By use of this method, the activity of MgsA was confirmed in the transformed host cells. (FIG. 9, FIG. 12).

Both GldA and SynADH are NADPH-dependent enzymes (FIG. 3). Therefore, the NADPH level was measured to determine the combined activity of the GldA and SynADH enzymes. The following method was used: Frozen pelleted cells were suspended in imidazole buffer (40 mM, pH 7) and total protein was extracted by grinding with glass beads at 30 Hz for 10 minutes. NADPH (200 μM) was added to the protein cell extract. Addition of 10 mM methylglyoxal started the reaction for combined GldA/synAKR activity or activity of SynADH alone, respectively. The change in OD₃₄₀ (maximum absorbance of NADPH) was measured to indicate enzyme activity.

The activity of MgsA or GldA/SynADH was then determined, as shown in FIG. 9. It was found that when the in vitro reaction was started with the substrate DHAP for the first step in the reaction, strain #748 had a high level of MgsA activity (about 3,000 nmol/mg protein*minute). When the same reaction was started with the intermediate substrate methylglyoxal, instead, the strains showed GldA/SynADH activity: #747: about 275 nmol/mg protein*minute; #748: about 610 nmol/mg protein*minute (FIG. 10).

### Example 7

### Determination of Enzymatic Activity of the SynAKR Enzyme

The NADPH-dependent enzyme synAKR is capable of catalyzing the conversion of methylglyoxal to lactaldehyde. The following method was used to measure the activity of synAKR. Frozen pelleted cells were suspended in imidazole buffer (40 mM, pH 7) and total protein was extracted by grinding with glass beads at 30 Hz for 10 minutes. NADPH (200 μM) was added to the protein cell extract. Methylglyoxal (10 mM) was added to start the reaction. The change in the absorption OD₃₄₀ (maximum absorbance of NADPH) was measured to indicate enzyme activity.

### Example 8

### Determination of Enzymatic Activity of the FucO Enzyme

The enzyme FucO is capable of catalyzing the NADPH-dependent conversion of hydroxyacetone to 1,2-propanediol. To determine the enzymatic activity of FucO, the following method was used. Frozen pelleted cells were suspended in imidazole buffer (40 mM, pH 7) and total protein was extracted by grinding with glass beads at 30 Hz for 10 minutes. NADPH (200 μM) was added to the protein cell extract. Hydroxyacetone (10 mM) was added to start the reaction. The change in the absorption OD₃₄₀ (maximum absorbance of NADPH) was measured to indicate enzyme activity.

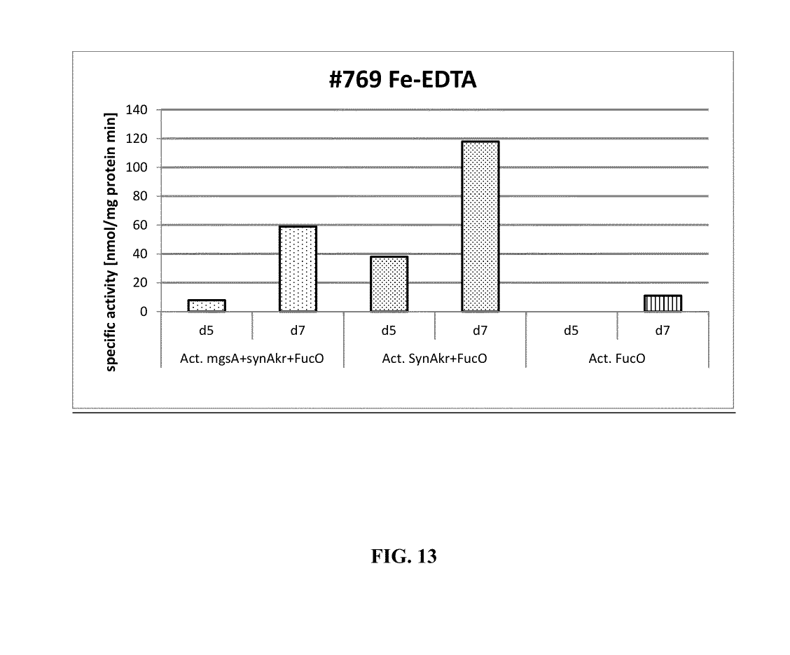

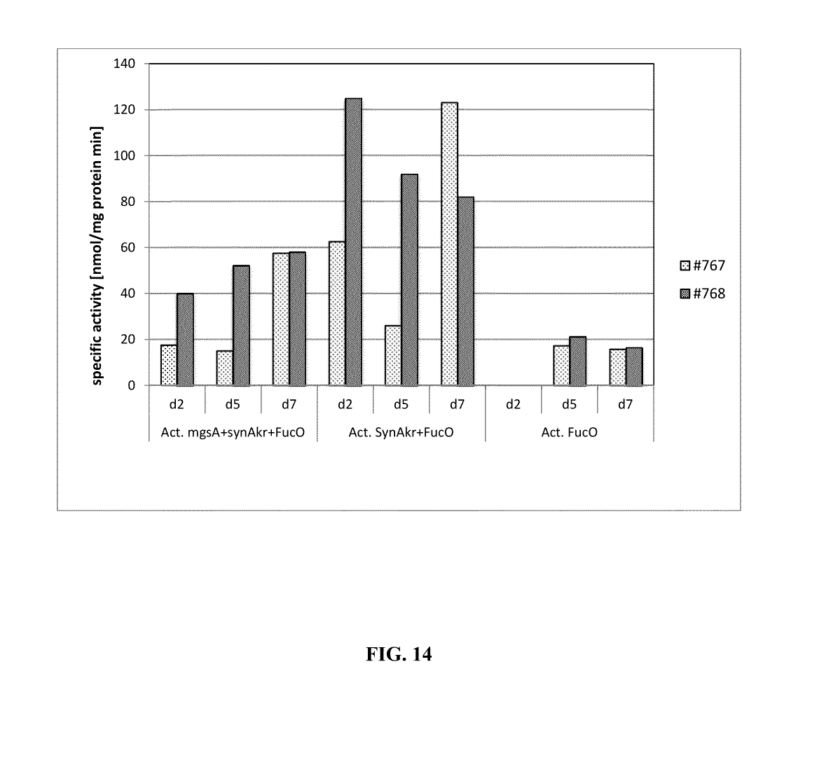

By use of this method, the activity of the enzyme FucO was confirmed in the host cells transformed with the FucO gene. Additionally, the increased product from the combination of synAKR and FucO or MgsA, SynAKR, and FucO was shown (FIG. 13, FIG. 14).

### Example 9

### Calculations of Enzymatic Activity

The activity of each of the enzymes described above (Examples 6-8) was determined as follows. The maximum slope in the linear area (ΔE) was determined based on the detected change in extinction over time. The protein concentration was determined by the Lowry method in order to calculate the specific activity, as shown below.

### Beer-Lambert-Law:

Eλ=ελ*c*d

where:

ελ: molar Extinction coefficient of sample at specific wavelength λ

c: concentration of sample

d: layer thickness (here d=1 cm)

Thus the change of extinction over time (ΔE) is:

ΔE=Eλ*min⁻¹=ελ*c*d*min⁻¹

This leads to the change of concentration over time c*min⁻¹

c*min⁻¹=ΔE*ελ

−1*d⁻¹*min⁻¹

Considering the dilution factor b (Volume of sample/Volume of used cell extract) and the overall protein concentration cₒᵥ, leads to the specific activity A_{spec}

A_{spec}=c*min⁻¹*b*cₒᵥ

−1

The basal specific activities of the wild type cells on the corresponding day were subtracted from each value. The resulting enzyme activity measurements are shown in FIGS. 9-10 and 12-14.

### Example 10

### Construction of Polycistronic Plasmids for 1,2-Propanediol Production in Cyanobacteria

The gene cassettes for 1,2-propanediol production shown in Example 3, above, contain genes that were each regulated by their own upstream promoter. To determine whether the genes could be regulated by just one upstream promoter controlling expression of several or all of the pathway genes, several polycistronic gene cassette arrangements were also prepared and tested, as detailed below.

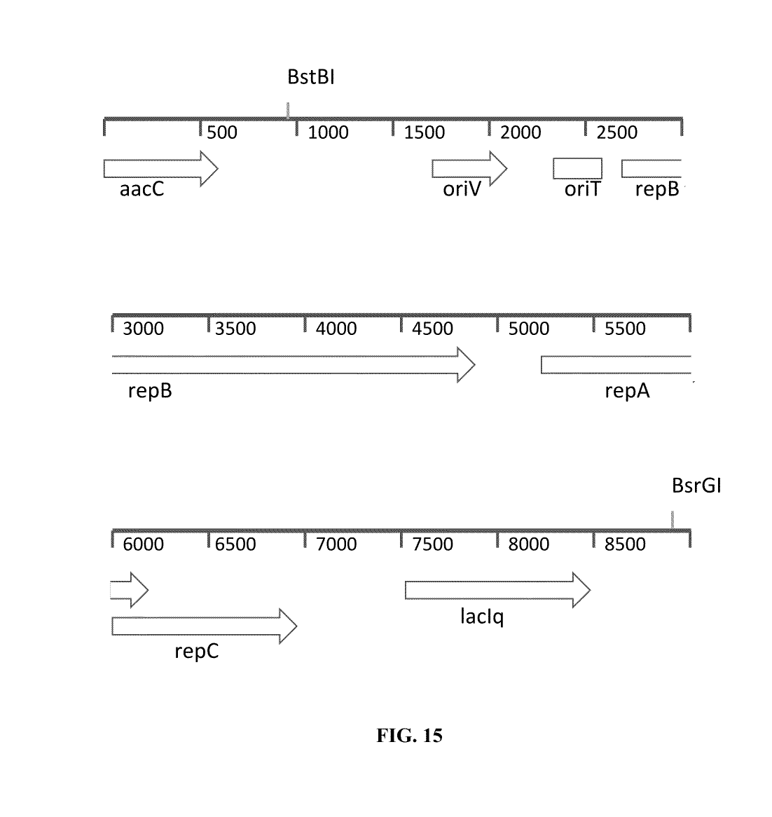

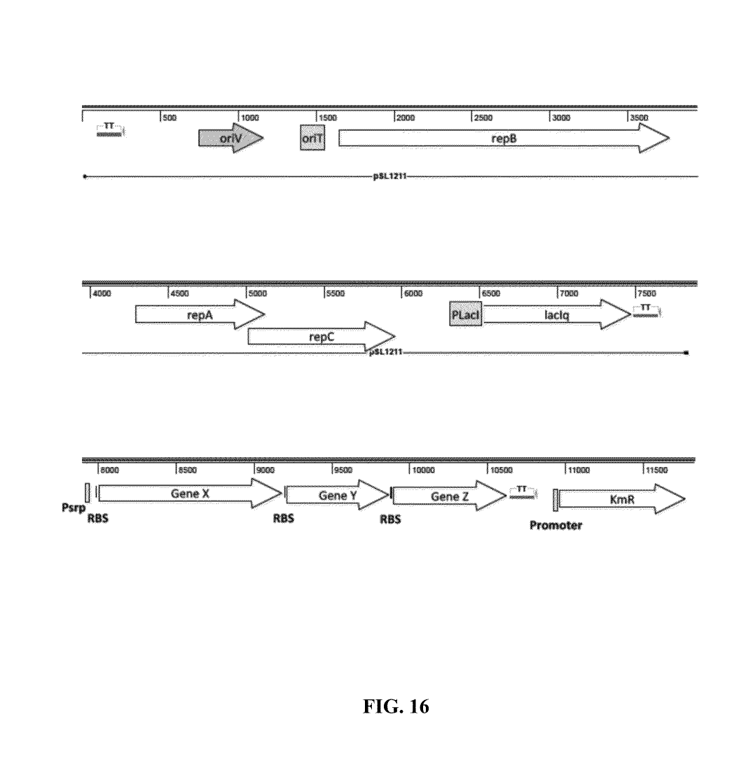

Many of the broad-host range plasmids described herein are derived from the RSF1010-derivative plasmid pSL1211, as shown in FIG. 15. An IPTG-inducible srp promoter and a kanamycin resistance gene were ligated into pSL1211, generating the plasmid pABb, to be used as a backbone plasmid for the heterologous expression of propanediol genes (FIG. 16).

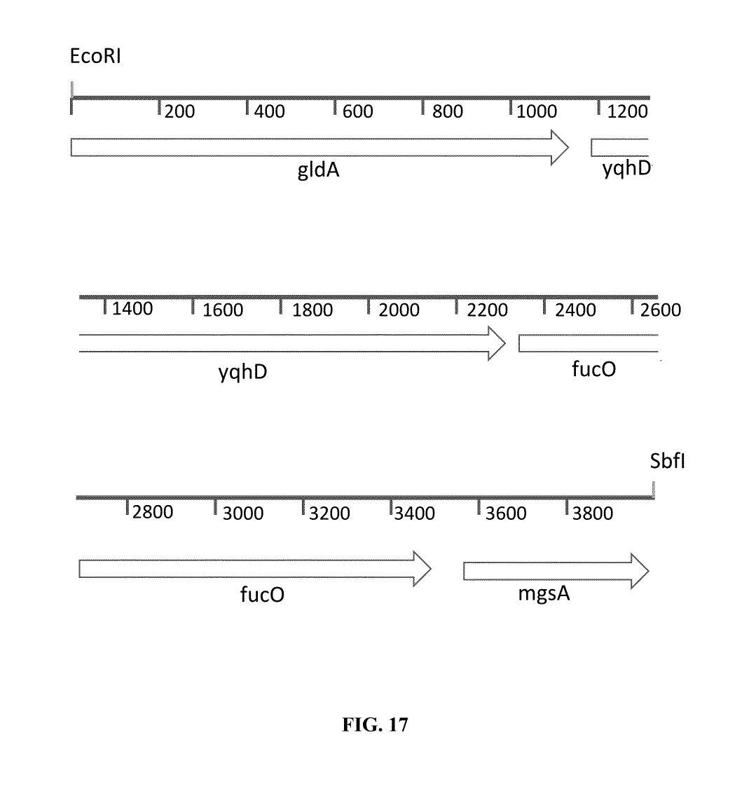

To determine whether all of the 1,2-propanediol pathway genes could be regulated by just one upstream promoter controlling expression of all of the pathway genes, a gene cassette having the following four enzymes (gldA-yqhD-fucO-mgsA), designed to have polycistronic expression driven by the Psrp promoter in a single operon, was prepared as shown in FIG. 17.

Each of the genes had its own RBS (ribosome-binding site). The genes were inserted into an RSF1010-derived plasmid backbone, as shown in Table 5. One construct was termed “pAB 1025”. The genes were amplified from wild type E. coli using the primers listed below in Table 4, following the manufacturer's protocol for Phusion polymerase. Overlap PCR was used to combine gldA and yqhD into a single PCR product and to combine fucO and mgsA into a single PCR product. These were ligated into TOPO blunt cloning vectors according to the manufacturer's instructions (Invitrogen, Carlsbad, Calif., USA). The TOPO vector containing gldA-yqhD was digested with NheI/SpeI, while the genes fucO-mgsA were digested out of the TOPO vector with Acc65I/Bpu10I. These were combined in a standard ligation reaction to make a TOPO-based plasmid containing the full operon, named pAB1012. The operon was amplified from pAB1012 by PCR with primers gldA F5 and mgsA R5 and inserted into the pABb plasmid (FIG. 3) digested with EcoRI/SbfI in a standard SLIC reaction to create pAB1025.

Additional plasmid constructs pAB1030 (SEQ ID NO: 44) and pAB1068 (SEQ ID NO: 45), differing only by the choice of promoter, were also prepared. These were constructed by amplifying the operon with primers which had the appropriate 5′ ends for inserting the PCR product into the appropriate vector by SLIC or by recombination cloning using GENEART Seamless Cloning and Assembly Kit from Invitrogen (Carlsbad, Calif., USA). Plasmid pAB1030, which was the same as pAB1025 except for the presence of a PnblA₇₁₂₀ promoter rather than Psrp to drive the 1,2-propanediol gene expression, was prepared as described above, except the vector used was pAB412, an RSF1010-derived plasmid containing a spectinomycin selection marker and a gene cassette (ZmPDC-SynADH) under the control of an nblA₇₁₂₀ promoter. The pAB412 vector was digested with EcoRI and PstI to remove the existing ZmPDC-SynADH gene cassette which created ends compatible for the previously described SLIC reaction. Plasmid pAB1068, which was the same as the pAB1025 plasmid except that it contained an smtA₇₀₀₂ promoter to drive the expression of the 1,2-propanediol genes rather than an srp promoter, was prepared as described above, except the vector used was pAB421, an RSF 1010-derived plasmid containing a gentamycin and spectinomycin resistance selection markers and a gene cassette (ZmPDC-SynADH) under the control of the smtA₇₀₀₂ promoter. The pAB421 vector was digested with EcoRI and PstI to remove the existing ZmPDC-SynADH gene cassette which created ends compatible for the previously described SLIC reaction.

TABLE 4
Primers for Construction of 1,2-Propanediol-Producing Plasmids

| Primer name | PCR Primer sequences |
|---|---|
| gldA F5 | aatgtgtggatcagcaggacgcactgaccgGAATTCGGCGCGCCagaggagaaCTTAAGatg |
| | gaccgcattattcaatcac |
| | (SEQ ID NO: 32) |
| gldA R5 | ttgttcatatgtagatctcctGTTAATTAAttattcccactcttgcaggaaac |
| | (SEQ ID NO: 33) |
| yqhD F5 | agtgggaataaTTAATTAACaggagatctacatatgaacaactttaatctgcacacc |
| | (SEQ ID NO: 34) |
| yqhD R5 | agccatcatGCTAGCtctcctcGGCCGGCCgcttagcgggcggcttcg |
| | (SEQ ID NO: 35) |
| fucO F5 | gcccgctaaGGCCGGCCgaggagaGCTAGCatgatggctaacagaatgattctg |
| | (SEQ ID NO: 36) |
| fucO R5 | cagttccatGCTAGCtctcctcGGCCGGCCttaccaggcggtatggtaaagc |
| | (SEQ ID NO: 37) |
| mgsA F5 | gcctggtaaGGCCGGCCgaggagaGCTAGCatggaactgacgactcgcac |
| | (SEQ ID NO: 38) |
| mgsA R5 | cgctactgccgccaggcaaattctgtttccTGCAGGCGCGCCttacttcagacggtccgcga |
| | (SEQ ID NO: 39) |
| gldA R | GGCACTGGCTGAACTGTGCTACAA |
| | (SEQ ID NO: 40) |
| fucO L | ACTTGCGCCGTTTCTCTTCGTC |
| | (SEQ ID NO: 41) |
| SpcF3 | CTCGGGCATCCAAGCAGC |
| | (SEQ ID NO: 42) |
| SpcF | GTAGAGCTATTCACTTTAGGTTTAG |
| | (SEQ ID NO: 43) |

### Example 11

### Confirmation of Plasmid Sequences and 1,2-Propanediol Production in E. coli

Several plasmid constructs (pAB1025, pAB1030 (SEQ ID NO: 44), pAB1061, pAB1062, pAB1068 (SEQ ID NO: 45), pAB1012, pAB1007, and pAB1008), each having the 1,2-propanediol pathway genes described in Example 10 were prepared and confirmed. The constructs differed in the choice of promoter, E. coli origin of replication, and cyanobacterial origin of replication, as shown below in Table 5. The sequence of the above-described plasmid pAB 1025 was confirmed by digestion with the restriction enzyme Avail and by sequencing. Plasmid pAB1030 was confirmed by digestion with the restriction enzyme BamHI and by sequencing. Plasmid pAB 1068 was confirmed by digestion with the restriction enzyme XmnI and by sequencing. The ability of the plasmid to produce 1,2-propanediol was first confirmed in E. coli. Once propanediol production in E. coli was confirmed, the plasmids were ready for transformation to cyanobacteria.

TABLE 5
1,2-Propanediol Polycistronic Plasmids

| | | | | Cyano- |
|---|---|---|---|---|
| | | | E coli | bacterial |
| Plasmid | | | Origin of | Origin of |
| Name | Promoter | Gene Cassette | Replication | Replication |
| pAB1025 | Psrp | gldA-yqhD- | RSF1010 | RSF1010 |
| | | fucO-mgsA | | |
| pAB1030 | PnblA₇₁₂₀ | gldA-yqhD- | RSF1010 | RSF1010 |
| | | fucO-mgsA | | |
| pAB1061 | ziaR-PziaA₆₈₀₃ | gldA-yqhD- | RSF1010 | RSF1010 |
| | | fucO-mgsA | | |
| pAB1062 | PsmtA₇₀₀₂ | gldA-yqhD- | pUC | pAQ1 |
| | | fucO-mgsA | | |
| pAB1068 | PsmtA₇₀₀₂ | gldA-yqhD- | RSF1010 | RSF1010 |
| | | fucO-mgsA | | |
| pAB1012 | Plac | gldA-yqhD- | pBR | N/A |
| | | fucO-mgsA | | |
| pAB1007 | Plac | gldA-yqhD | pBR | N/A |
| pAB1008 | Plac | fucO-mgsA | pBR | N/A |

### Example 12

### Transformation of Plasmids Harboring the Polycistronic 1,2-Propanediol-Producing Genes to Cyanobacteria

Cyanobacterial strains Synechococcus sp. PCC 7002 and Synechocystis sp. PCC 6803 were transformed with the plasmids harboring the 1,2-propanediol-producing genes. Synechocystis sp. PCC 6803 was transformed with plasmids pAB1025, pAB1030, and pAB1068. Synechococcus sp. PCC 7002 was transformed with plasmids pAB1025, pAB1030, and pAB1068. Transformation procedures were performed via conjugation as follows. One week before the day of conjugation, cyanobacterial cells (e.g. PCC 7002 and PCC 6803) were inoculated with a fresh culture using a ˜1:10 dilution of an older (1 week) culture. E. coli cultures containing the plasmid(s) of interest and the helper plasmid pRL443 (described in Elhai et al. (1997), Journal of Bacteriology, 179:1998-2005) were started the night before the planned conjugation in ˜3 ml LB supplemented with the appropriate antibiotic(s). Four hours prior to conjugation, 30 ml of fresh LB medium (with appropriate antibiotic(s)) was inoculated with ˜0.5 ml of the overnight culture. The E. coli and cyanobacterial cultures were transferred to a 50 ml conical tube and centrifuged at 2,500×g for 10 minutes at room temperature to pellet the cells. The supernatant was decanted, and the cell pellets were resuspended in 1 ml LB (for the E. coli cultures) or (M)BG-11 (for cyanobacteria). The cells were then transferred to a microcentrifuge tube and centrifuged at 2,500×g for 10 minutes at room temperature. The decanting, resuspension, and centrifuge steps were repeated, resuspending each pellet in 300 μl LB or (M)BG-11, as appropriate. The cell resuspensions were diluted and the cells were counted.

The next step involved preparing a mixture of cyanobacterial cells, the helper plasmid, and the plasmids harboring the 1,2-propanediol-producing genes in about a 1:1:1 cell count ratio. Approximately 3.6×10⁸ cells each of cyanobacteria, E. coli with plasmid pRL443, and E. coli with the plasmid of interest was placed in a microcentrifuge tube. The cell mixture was then centrifuged at 2,500×g for 5 minutes at room temperature. The supernatant was decanted and the pellet was resuspended in 950 μl (M)BG-11 and 50 μl LB. Sterilized cellulose nitrate membrane filters (Whatman) were transferred to (M)BG-11 (vB₁₂)+5% LB agar plates. A 200 μl aliquot of the mixture was spread evenly on the filter. The agar plate was then placed in low light for two days. The filter was then transferred onto a fresh (M)BG-11 (vB₁₂) agar plate containing the appropriate selective antibiotic. MBG-11 vB₁₂ plates had the following final antibiotic concentrations: spectinomycin, 100 μg/ml; kanamycin, 40 μg/ml. BG-11 plates had the following final antibiotic concentrations: spectinomycin, 15 μg/ml; kanamycin, 10 μg/ml. After 8-12 days, the presence of single colonies on the filters was monitored. Once single colonies were observed, the colonies were streaked onto a fresh selective plate (1st pass plate). The process was repeated (2nd pass plate). Once colonies were observed on the 2nd pass plate, the patch was taken and streaked onto an LB plate to check for potential E. coli contamination. Clean patches were used to perform colony PCR to test for the plasmid of interest.

### Example 13

### Colony PCR for Verification of Transformation

To confirm the presence of the 1,2-propanediol genes in the cyanobacterial host cells, streaks from colonies were resuspended in TE buffer (MediaTech, Inc, Manassas, Va., USA, and the cells were disrupted with glass beads. The supernatants were used as a DNA template for PCR amplifications of fragments of the 1,2-propanediol genes using the gldA R and fucO L primers. The results of the PCR analysis confirmed the presence of the 1,2-propanediol genes in the host cells.

Cells from verified streaks were then used to inoculate 3 ml liquid BG-11 or MBG-11 vB₁₂ cultures supplemented with the appropriate antibiotics (MBG-11 vB₁₂ medium had the following final antibiotic concentrations: spectinomycin, 100 μg/ml; kanamycin, 40 μg/ml; BG-11 medium had the following final antibiotic concentrations: spectinomycin, 15 μg/ml; kanamycin, 10 μg/ml) and incubated under a light intensity of 10-20 μmol m⁻² s⁻¹ at 37° C.

### Example 14

### Extraction and Detection of 1,2-Propanediol

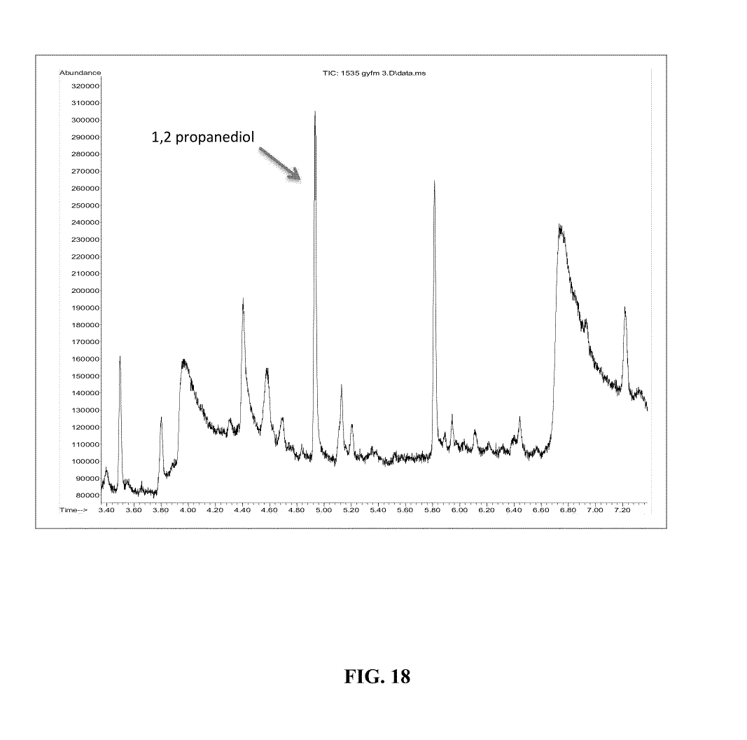

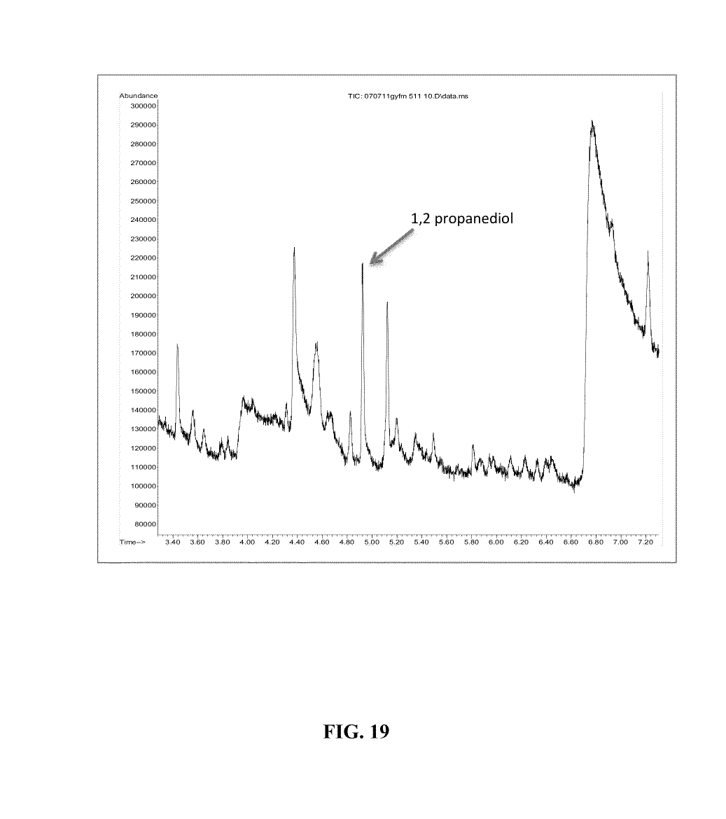

A methanol/phosphate extraction was used to separate 1,2-propanediol produced from the culture. Five ml of cyanobacterial culture was saturated with dipotassium phosphate (˜6 g). This mixture was amended with methanol to a final methanol concentration of 30%, and was then vigorously shaken three times with five minute rest intervals. This extraction was left overnight at room temperature to allow phase separation. The upper methanol layer was collected avoiding the interface and evaporated to ˜100 μl (15× concentration) in a benchtop centrifugal evaporator. This extract was passed through a 0.2 μm filter prior to analysis. The methanol extract was loaded onto a GC/MS using a liquid injection. 1,2-propanediol was measured using gas chromatography with flame ionization detection. A Stabilwax column (30 m length, 0.53 mm diameter, 1 μm film) was used on an Agilent 7890A GC system equipped with a 7683B liquid injector. A cyclo-uniliner was installed on the split/splitless injector and heated to 225° C. Two microliters were injected using a pulsed splitless program at 10 psi for 0.1 min. Using helium as the carrier gas at 50 cm/sec, separation was performed by running a linear thermal program from 80° C. to 200° C. at 24° C./min with a 5 minute hold at 200° C. Using this method, the retention time of 1,2-propanediol was 4.9 minutes. The cyanobacterial Synechococcus sp. PCC 7002 transformed with the plasmid pAB1025 produced ˜1 μM or 72 μg/L 1,2-propanediol, as shown in FIG. 18. The cyanobacterial strain Synechocystis sp. PCC 6803 transformed with plasmid pAB1025 produced ˜2.5 μM or 0.2 mg/L 1,2-propanediol, as shown in FIG. 19.

### Example 15

### Production of S-1,2-Propanediol

In a prophetic example, S-1,2-propanediol can be produced by following certain portions of the propanediol pathways shown herein. As shown in FIG. 2, the 1,2-propanediol intermediate acetol can be converted to the S form of 1,2-propanediol. Further, as demonstrated in Example 8, the enzyme FucO is capable of catalyzing the NADPH-dependent conversion of hydroxyacetone to 1,2-propanediol. In this prophetic example, a host cyanobacterial cell is transformed with a shuttle vector containing a plasmid carrying genes encoding the enzymes MgsA, methylglyoxyl reductase, and FucO. The production of S-1,2-propanediol is confirmed by chemical analysis. By use of this method, S-1,2-propanediol is produced in cyanobacteria.

### Example 16

### Tolerance Testing for Determination of Suitable Cyanobacterial Strains for 1,2-Propanediol Production

The tolerance of cyanobacterial strains PCC 6803 and PCC 7002 to the presence of accumulated 1,2-propanediol in the culture medium was examined by adding a one time bolus of varying amounts of 1,2-propanediol (1%, 2%, 3% and 5%) to exponential phase cultures and comparing the growth of these cultures to a wild type culture with no addition. Growth was monitored by optical density (OD₇₅₀) for one week. There was no difference in the growth of cultures containing up to 3% 1,2-propanediol compared to wild type for either strain. The addition of 5% 1,2-propanediol was found to inhibit Synechocystis sp. PCC 6803 causing less growth, discoloration and clumping, but the culture did not bleach out and die. The same effect was observed with the addition of 5% 1,2-propanediol to Synechococcus sp. PCC 7002. No lethal effect (complete bleaching) was observed within these parameters.

### Example 17

### Production of 1,2-Propanediol from a Svnechococcus Culture in a 500 Liter Outdoor Photobioreactor

A strain of Synechococcus PCC 7002 cells modified to contain a 1,2-propanediol gene cassette is inoculated into a 500 L enclosed outdoor photobioreactor in seawater containing BG-11 nutrients and vitamin B₁₂ (1 μg/ml) and grown for three months. Every two weeks, 50% of the culture medium is separated from the remaining cells and removed from the culture, and fresh replacement medium is added to the photobioreactor. The spent culture medium is filtered, pH treated, flocculated, filtered once again, then the resulting liquid is treated with a distillation procedure to result in substantially purified 1,2-propanediol. Following this method, a healthy, continuously growing cyanobacterial culture is able to produce 1,2-propanediol continuously for a range of time from about several months, to a year or more.

Although the present invention has been described in considerable detail with reference to certain embodiments thereof, other embodiments are possible. Therefore, the spirit and scope of the appended claims should not be limited to the description of the embodiments contained therein.

What is claimed is:

1. A genetically enhanced cyanobacterial cell, comprising:
a) at least one promoter capable of regulating gene expression in cyanobacteria; and
b) a gldA gene, a fucO gene, and an mgsA gene,
wherein said at least one promoter is operably linked to said gldA, fucO, and mgsA genes, further wherein said cell produces 1,2-propanediol.

2. The genetically enhanced cyanobacterial cell of claim 1, further comprising a yqhD gene.

3. The genetically enhanced cyanobacterial cell of claim 2, wherein at least one of said genes is present in a location selected from the group consisting of an exogenously derived extrachromosomal plasmid, an endogenous plasmid-derived extrachromosomal plasmid, and on the cyanobacterial chromosome.

4. The genetically enhanced cyanobacterial cell of claim 2, wherein said at least one promoter is selected from the group consisting of: Psrp, PnblA₇₁₂₀, PrbcL₆₈₀₃, PsmtA₇₀₀₂, and ziaR-PziaA₆₈₀₃.

5. The genetically enhanced cyanobacterial cell of claim 2, wherein the gldA gene has at least 98% identity to SEQ ID NO: 11.

6. The genetically enhanced cyanobacterial cell of claim 2, wherein the gldA gene encodes a polypeptide having at least 98% identity to SEQ ID NO: 12.

7. The genetically enhanced cyanobacterial cell of claim 2, wherein the fucO gene has at least 98% identity to SEQ ID NO: 13.

8. The genetically enhanced cyanobacterial cell of claim 2, wherein the fucO gene encodes a polypeptide having at least 98% identity to SEQ ID NO: 14.

9. The genetically enhanced cyanobacterial cell of claim 2, wherein the mgsA gene has at least 98% identity to SEQ ID NO: 15.

10. The genetically enhanced cyanobacterial cell of claim 2, wherein the mgsA gene encodes a polypeptide having at least 98% identity to SEQ ID NO: 16.

11. The genetically enhanced cyanobacterial cell of claim 2, wherein the yqhD gene has at least 98% identity to SEQ ID NO: 17.

12. The genetically enhanced cyanobacterial cell of claim 2, wherein the yqhD gene encodes a polypeptide having at least 98% identity to SEQ ID NO: 18.

13. The genetically enhanced cyanobacterial cell of claim 2, wherein said genes are located together under the regulation of one promoter.

14. The genetically enhanced cyanobacterial cell of claim 2, wherein at least one of the genes is present in a separate genetic region in the cell.

15. The genetically enhanced cyanobacterial cell of claim 14, wherein said separate genetic region in the cell is a different plasmid vector or a different chromosome.

16. The cyanobacterial cell of claim 2, wherein said cyanobacterial cell is selected from the group consisting of Synechocystis sp. PCC 6803 and Synechococcus sp. PCC 7002.

17. A method of producing 1,2-propanediol in a cyanobacterial cell, comprising culturing a genetically enhanced cyanobacterial cell of claim 2 under conditions wherein the cyanobacterial cell produces 1,2-propanediol.

18. A method of producing 1,2-propanediol in a cyanobacterial cell, comprising:
a) transforming said cell with an mgsA gene, a gene encoding an enzyme capable of converting methylglyoxal to lactaldehyde, and a gene encoding an enzyme capable of converting lactaldehyde to 1,2-propanediol, and
b) producing 1,2-propanediol from the cyanobacterial cell.

19. The method of claim 18, wherein said gene encoding an enzyme capable of converting methylglyoxal to lactaldehyde is selected from the group consisting of GldA, SynADH, and SynAKR.

20. The method of claim 18, wherein said gene encoding an enzyme capable of converting lactaldehyde to 1,2-propanediol is selected from FucO and GldA.

21. The method of claim 18, wherein each of said genes is under the control of a separate promoter.

22. The method of claim 18, wherein all of the said genes are under the control of one promoter.


SEQUENCE LISTING (54 sequences)

SEQ ID NO: 1 - DNA, 41 bp, Artificial Sequence
  Synthetic promoter sequence
ggcgaattgacattgtgagcggataacaatataatgtgtgg

SEQ ID NO: 2 - DNA, 604 bp, Nostoc sp. PCC7120
gataacatcaccgtcgttatcgtcgctttagaataacgttcccaaaatagctcatttccaactggcaactcacaaccaaaaaccgcatttttagtaaatatactcagcaatttgttcaacctgagcatttttcccatttgcaacttgatacaaatatttttagcagcaaattttcctactgccagcttagtttacataaattttgtctgttgacatcttgcacacaataaggtatggcgcatataatgcgatattactaccattaatttactacctagtcattaacgtctcccgccagagaacagttttgaataggtagtcaattttaggtattgaacctgctgtaaatttattaaatcgatgaatttccccgaaatctgctctagcagacttgggttatataccagtaggctcaggtgcaaaacaacaaagcacaaattttacccattaaggatataggcaatctgtcaaatagttgttatctttcttaatacagaggaataatcaacaatatggggcaggtactaactaaagtcctatgcctgtggggcttctgtaaccgacataacctttacgcgttgtcttttaggagtctgttatgaac

SEQ ID NO: 3 - DNA, 267 bp, Synechocystis sp. PCC6803
tcgacatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgacctagatg

SEQ ID NO: 4 - DNA, 253 bp, Synechocystis sp. PCC6803
atcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactg

SEQ ID NO: 5 - DNA, 65 bp, Synechocystis sp. PCC6803
cattgccataagtaaaggcatcccctgcgtgataagattaccttcagtttatggaggactgacca

SEQ ID NO: 6 - DNA, 59 bp, Synechocystis sp. PCC6803
attgccataagtaaaggcatcccctgcgtgataagattaccttcactaaaggagcaatt

SEQ ID NO: 7 - DNA, 123 bp, Synechococcus sp. PCC 7002
tcgactgtggtctgtctttgttcgctgatctaaacaatacctgaataattgttcatgtgttaatctaaaaatgtgaacaatcgttcaactatttaagacaataccttggaggtttaaaccatg

SEQ ID NO: 8 - DNA, 550 bp, Synechocystis sp. PCC 6803
ggcggccaacgtgatttaaagaaaaacctccttgaaccgtagcacaaatcttgaaacacctgaagatatgctcagatattaaagatgttaggatgaaaatcattttctaaatccagtttaaatttttccctagctcctaacgccaacctctatgagtaagtcctcgttgtcaaagtcacaatcctgccagaacgaagagatgcccctttgtgatcaacctcttgttcatcttgagcaggtacgacaggttcaaccagaggtgatgtcattggaccaggcccagcaaatggcggagtttttcagtgcactagctgatccgagtcggttgcgtttaatgtcggcattggcccgccaagaactctgtgtctgtgatttagcagcggcgatgaaagtgagtgaatcggcagtttcccatcaattacgaattttacgatcgcagcgcctggtaaagtatcgccgggtcggccgtaatgtttactacagcttggcggataatcatgtgatgaatttgtatcgggaagttgcagaccatttgcaggaatcggattaa

SEQ ID NO: 9 - DNA, 276 bp, Synechocystis PCC 6803
agggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggag

SEQ ID NO: 10 - DNA, 31 bp, Artificial Sequence
  synthetic terminator sequence derived from lambda phage
aacgctcggttgccgccgggcgttttttatt

SEQ ID NO: 11 - DNA, 1104 bp, Escherichia coli
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 12 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 13 - DNA, 1152 bp, Escherichia coli
atgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 14 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 15 - DNA, 459 bp, Escherichia coli
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 16 - protein, 152 aa, Escherichia coli [sequence omitted]

SEQ ID NO: 17 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 18 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 19 - DNA, 963 bp, Synechococcus PCC 7002
atgaaaacaagacaactaggccaaagtgccgtccaaatcaccccgattattctcggtacttggcaagcgggcaagcgcaattgggcggatattgacgaccaagaaattgtggccgggatccgtgccgccgtagatgcaggcattacgaccatcgataccgctgaaatttatggcgatggggattctgaacgtcgggtcgccgaggcgatcgccccccaacgggatcaagtgaccctattaacgaaagtctttgccaatcacctccaccacgaccaggtgatcaccgcctgcgaaaattccctcaacagactccagacagactacatcgatctgtaccaaatccactggccagcgggaacgtggaattctgacctggtgcccatcgctgaaaccatggccgctctgaatcaattgaaagaacagggcaaaattcgcgctattggtgtgtctaatttttccttggcgcaactccaggaagcgatggaacacggccaaatcgatagcattcaaccgccctattctttattttggcgggccattgaacgggaaattcaacctttctgtgcggcccagcagatttcgatcctcgcctattcttccttggcccagggtctactgacggggaaatttggccccgatcaccagtttgcggcgggggatcaccgctcccacaaccgtctttatgctgacccggaaaattaccaacgggtacaaacggccctcggactcctgaaaccgatcgccacgacaaagaattgcaccttggctcaactggcgatcgcctggctgattcggcagccccaaaccaatgccatcgtcggcgcgcgcaatgctcaacaggcgatcgccaatgcccaggccatcgatgtcgagttaacggctaaagatctcgaagccattgaccatatcgggcggacagtaaccgatcctctagacgaaaatccgctcctatggaactggtaa

SEQ ID NO: 20 - protein, 320 aa, Synechococcus PCC 7002
MKTRQLGQSAVQITPIILGTWQAGKRNWADIDDQEIVAGIRAAVDAGITTIDTAEIYGDGDSERRVAEAIAPQRDQVTLLTKVFANHLHHDQVITACENSLNRLQTDYIDLYQIHWPAGTWNSDLVPIAETMAALNQLKEQGKIRAIGVSNFSLAQLQEAMEHGQIDSIQPPYSLFWRAIEREIQPFCAAQQISILAYSSLAQGLLTGKFGPDHQFAAGDHRSHNRLYADPENYQRVQTALGLLKPIATTKNCTLAQLAIAWLIRQPQTNAIVGARNAQQAIANAQAIDVELTAKDLEAIDHIGRTVTDPLDENPLLWNW

SEQ ID NO: 21 - DNA, 1011 bp, Artificial Sequence
  ADH gene originally derived from Synechocystis PCC 6803; codon optimized for increased expression.
atgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactga

SEQ ID NO: 22 - protein, 336 aa, Synechocystis PCC6803
MIKAYAALEANGKLQPFEYDPGALGANEVEIEVQYCGVCHSDLSMINNEWGISNYPLVPGHEVVGTVAAMGEGVNHVEVGDLVGLGWHSGYCMTCHSCLSGYHNLCATAESTIVGHYGGFGDRVRAKGVSVVKLPKGIDLASAGPLFCGGITVFSPMVELSLKPTAKVAVIGIGGLGHLAVQFLRAWGCEVTAFTSSARKQTEVLELGAHHILDSTNPEAIASAEGKFDYIISTVNLKLDWNLYISTLAPQGHFHFVGVVLEPLDLNLFPLLMGQRSVSASPVGSPATIATMLDFAVRHDIKPVVEQFSFDQINEAIAHLESGKAHYRVVLSHSKN

SEQ ID NO: 23 - DNA, 12696 bp, Artificial Sequence
  Heterologous chimeric plasmid construct
atgaattcttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtttttggggatcaattcgagctcggtacccaaactagtcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagagtcgacgggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaacta

SEQ ID NO: 24 - DNA, 4403 bp, Artificial Sequence
  Heterologous chimeric plasmid construct
gatcttctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcgtggacagcaccctcggtttagaaattattgaagtcgtagaacaagcggcgatcgcctcggcaaaatggatgggcaaaggtgaaaaaaacaccgctgaccaagtagccgtagaagccatgcgggaacggatgaataaaatccacatgcggggccgcatcgtcattggcgagggggaaagggatgatgcacccatgctctacatcggcgaagaagtaggcatttgcaccagagaagatgccaaatccttctgcaatcccgacgaattggtagaaattgacattgctgttgacccctgtgaaggtaccaacctagtagcctatggtcaaaacggttccatggccgtgttggcaatttctgaaaaaggtggtttgtttgccgctcccgacttctacatgaaaaaactggcggctcccccagcggccaaaggtcatgtggacatcgacaaatcggccaccgaaaacctgaaaatcctctccgattgcctcaaccgcagcattgaagaattggtggtagtggtcatggatcgtccccgccacaaagaattgatccaagaaatccgcaatgccggtgctcgggtacgtctaatcagtgatggagacgtttccgccgccatttcctgtgctttttccggcaccaacatccacgctctgatgggcatcggggctgctcctgaaggggtaatttccgctgctgccatgcgctgtctggggggtcacttccaaggtcagttaatctacgatcccgaagtggttaaaaccggcctaatcggtgaaagtcgggaaggcaacctagagcgtctcgcttccatgggcatcaaaaatcctgaccaggtttataactgcgaagaattggcctgtggcgaaaccgtattatttgccgcctgtggtatcacccctggcactttgatggaaggagtccgcttcttccatggtggggtacggacccaaagcttggttatttctagccagtccagcaccgcccgctttgtagacactgtccatatgaaggaaagccccaaagtaatccaactgcattaatttccctctcggcaactatccccagaaggttaaaacctggggaaatacatcgccgtttcccttgtcccctgaagttcagactttgggggacttatgttttggttgggtcactagtatcttgctgaaaaactcgagccatccggaagatctggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtagga

SEQ ID NO: 25 - DNA, 3499 bp, Artificial Sequence
  Heterologous chimeric plasmid construct
gaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgagatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccgcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagat

SEQ ID NO: 26 - DNA, 4392 bp, Artificial Sequence
  Heterologous chimeric plasmid construct
aaagatatctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcgtggacagcaccctcggtttagaaattattgaagtcgtagaacaagcggcgatcgcctcggcaaaatggatgggcaaaggtgaaaaaaacaccgctgaccaagtagccgtagaagccatgcgggaacggatgaataaaatccacatgcggggccgcatcgtcattggcgagggggaaagggatgatgcacccatgctctacatcggcgaagaagtaggcatttgcaccagagaagatgccaaatccttctgcaatcccgacgaattggtagaaattgacattgctgttgacccctgtgaaggtaccaacctagtagcctatggtcaaaacggttccatggccgtgttggcaatttctgaaaaaggtggtttgtttgccgctcccgacttctacatgaaaaaactggcggctcccccagcggccaaaggtcatgtggacatcgacaaatcggccaccgaaaacctgaaaatcctctccgattgcctcaaccgcagcattgaagaattggtggtagtggtcatggatcgtccccgccacaaagaattgatccaagaaatccgcaatgccggtgctcgggtacgtctaatcagtgatggagacgtttccgccgccatttcctgtgctttttccggcaccaacatccacgctctgatgggcatcggggctgctcctgaaggggtaatttccgctgctgccatgcgctgtctggggggtcacttccaaggtcagttaatctacgatcccgaagtggttaaaaccggcctaatcggtgaaagtcgggaaggcaacctagagcgtctcgcttccatgggcatcaaaaatcctgaccaggtttataactgcgaagaattggcctgtggcgaaaccgtattatttgccgcctgtggtatcacccctggcactttgatggaaggagtccgcttcttccatggtggggtacggacccaaagcttggttatttctagccagtccagcaccgcccgctttgtagacactgtccatatgaaggaaagccccaaagtaatccaactgcattaatttccctctcggcaactatccccagaaggttaaaacctggggaaatacatcgccgtttcccttgtcccctgaagttcagactttgggggacttatgttttggttgggtcactagtatcttgctgaaaaactcgagccatccggaagatctggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatcttctag

SEQ ID NO: 27 - DNA, 3749 bp, Artificial Sequence
  Heterologous chimeric plasmid construct
gatcttctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgagccatccggaagatctggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtagga

SEQ ID NO: 28 - DNA, 3738 bp, Artificial Sequence
  Heterologous chimeric plasmid construct
aaagatatctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgagccatccggaagatctggcggccgctctccctatagtgagtcgtattacgccggatggatatggtgttcaggcacaagtgttaaagcagttgattttattcactatgatgaaaaaaacaatgaatggaacctgctccaagttaaaaatagagataataccgaaaactcatcgagtagtaagattagagataatacaacaataaaaaaatggtttagaacttactcacagcgtgatgctactaattgggacaattttccagatgaagtatcatctaagaatttaaatgaagaagacttcagagcttttgttaaaaattatttggcaaaaataatataattcggctgcaggggcaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcaattggcagtgagcgcaacgcaattaatgtgagttagctcactcattaggcaccccaggctttacactttatgcttccggctcgtataatgtgtggaattgtgagcggataacaatttcacacaggaggtttaaactttaaacatgtcaaaagagacgtcttttgttaagaatgctgaggaacttgcaaagcaaaaaatggatgctattaaccctgaactttcttcaaaatttaaatttttaataaaattcctgtctcagtttcctgaagcttgctctaaacctcgttcaaaaaaaatgcagaataaagttggtcaagaggaacatattgaatatttagctcgtagttttcatgagagtcgattgccaagaaaacccacgccacctacaacggttcctgatgaggtggttagcatagttcttaatataagttttaatatacagcctgaaaatcttgagagaataaaagaagaacatcgattttccatggcagctgagaatattgtaggagatcttctag

SEQ ID NO: 29 - DNA, 4008 bp, Artificial Sequence
  Heterologous chimeric plasmid construct
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatacatatgacccgccagaaaaacgagcttatgaaaacaagacaactaggccaaagtgccgtccaaatcaccccgattattctcggtacttggcaagcgggcaagcgcaattgggcggatattgacgaccaagaaattgtggccgggatccgtgccgccgtagatgcaggcattacgaccatcgataccgctgaaatttatggcgatggggattctgaacgtcgggtcgccgaggcgatcgccccccaacgggatcaagtgaccctattaacgaaagtctttgccaatcacctccaccacgaccaggtgatcaccgcctgcgaaaattccctcaacagactccagacagactacatcgatctgtaccaaatccactggccagcgggaacgtggaattctgacctggtgcccatcgctgaaaccatggccgctctgaatcaattgaaagaacagggcaaaattcgcgctattggtgtgtctaatttttccttggcgcaactccaggaagcgatggaacacggccaaatcgatagcattcaaccgccctattctttattttggcgggccattgaacgggaaattcaacctttctgtgcggcccagcagatttcgatcctcgcctattcttccttggcccagggtctactgacggggaaatttggccccgatcaccagtttgcggcgggggatcaccgctcccacaaccgtctttatgctgacccggaaaattaccaacgggtacaaacggccctcggactcctgaaaccgatcgccacgacaaagaattgcaccttggctcaactggcgatcgcctggctgattcggcagccccaaaccaatgccatcgtcggcgcgcgcaatgctcaacaggcgatcgccaatgcccaggccatcgatgtcgagttaacggctaaagatctcgaagccattgaccatatcgggcggacagtaaccgatcctctagacgaaaatccgctcctatggaactggtaagcaccaacgctcggttgccgccgggcgttttttattcctgcagatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 30 - DNA, 4199 bp, Artificial Sequence
  Heterologous chimeric plasmid construct
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatcccgggattgccataagtaaaggcatcccctgcgtgataagattaccttcactaaaggagcaattatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataacctactccaaactcccggcttgtccgggagtttgaacgcaaaattgcctgtctagaatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 31 - DNA, 4236 bp, Artificial Sequence
  Heterologous chimeric plasmid construct
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatcccgggattgccataagtaaaggcatcccctgcgtgataagattaccttcaacaaggagaaggatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaatgcgctgatgaacgctcggttgccgccgggcgttttttattctagaatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattgttatccgctcacaattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 32 - DNA, 81 bp, Artificial Sequence
  Synthetic PCR Primer Sequence
aatgtgtggatcagcaggacgcactgaccggaattcggcgcgccagaggagaacttaagatggaccgcattattcaatcac

SEQ ID NO: 33 - DNA, 53 bp, Artificial Sequence
  Synthetic PCR Primer Sequence
ttgttcatatgtagatctcctgttaattaattattcccactcttgcaggaaac

SEQ ID NO: 34 - DNA, 57 bp, Artificial Sequence
  Synthetic PCR Primer Sequence
agtgggaataattaattaacaggagatctacatatgaacaactttaatctgcacacc

SEQ ID NO: 35 - DNA, 48 bp, Artificial Sequence
  Synthetic PCR Primer Sequence
agccatcatgctagctctcctcggccggccgcttagcgggcggcttcg

SEQ ID NO: 36 - DNA, 54 bp, Artificial Sequence
  Synthetic PCR Primer Sequence
gcccgctaaggccggccgaggagagctagcatgatggctaacagaatgattctg

SEQ ID NO: 37 - DNA, 52 bp, Artificial Sequence
  Synthetic PCR Primer Sequence
cagttccatgctagctctcctcggccggccttaccaggcggtatggtaaagc

SEQ ID NO: 38 - DNA, 50 bp, Artificial Sequence
  Synthetic PCR Primer Sequence
gcctggtaaggccggccgaggagagctagcatggaactgacgactcgcac

SEQ ID NO: 39 - DNA, 62 bp, Artificial Sequence
  Synthetic PCR Primer Sequence
cgctactgccgccaggcaaattctgtttcctgcaggcgcgccttacttcagacggtccgcga

SEQ ID NO: 40 - DNA, 24 bp, Artificial Sequence
  Synthetic PCR Primer Sequence
ggcactggctgaactgtgctacaa

SEQ ID NO: 41 - DNA, 22 bp, Artificial Sequence
  Synthetic PCR Primer Sequence
acttgcgccgtttctcttcgtc

SEQ ID NO: 42 - DNA, 18 bp, Artificial Sequence
  Synthetic PCR Primer Sequence
ctcgggcatccaagcagc

SEQ ID NO: 43 - DNA, 25 bp, Artificial Sequence
  Synthetic PCR Primer Sequence
gtagagctattcactttaggtttag

SEQ ID NO: 44 - DNA, 12667 bp, Artificial Sequence
  heterologous chimeric plasmid construct
aacgatctgatagagaagggtttgctcgggtcggtggctctggtaacgaccagtatcccgatcccggctggccgtcctggccgccacatgaggcatgttccgcgtccttgcaatactgtgtttacatacagtctatcgcttagcggaaagttcttttaccctcagccgaaatgcctgccgttgctagacattgccagccagtgcccgtcactcccgtactaactgtcacgaacccctgcaataactgtcacgcccccctgcaataactgtcacgaacccctgcaataactgtcacgcccccaaacctgcaaacccagcaggggcgggggctggcggggtgttggaaaaatccatccatgattatctaagaataatccactaggcgcggttatcagcgcccttgtggggcgctgctgcccttgcccaatatgcccggccagaggccggatagctggtctattcgctgcgctaggctacacaccgccccaccgctgcgcggcagggggaaaggcgggcaaagcccgctaaaccccacaccaaaccccgcagaaatacgctggagcgcttttagccgctttagcggcctttccccctacccgaagggtgggggcgcgtgtgcagccccgcagggcctgtctcggtcgatcattcagcccggctcatccttctggcgtggcggcagaccgaacaaggcgcggtcgtggtcgcgttcaaggtacgcatccattgccgccatgagccgatcctccggccactcgctgctgttcaccttggccaaaatcatggcccccaccagcaccttgcgccttgtttcgttcttgcgctcttgctgctgttcccttgcccgcacccgctgaatttcggcattgattcgcgctcgttgttcttcgagcttggccagccgatccgccgccttgttgctccccttaaccatcttgacaccccattgttaatgtgctgtctcgtaggctatcatggaggcacagcggcggcaatcccgaccctactttgtaggggagggcgcacttaccggtttctcttcgagaaactggcctaacggccacccttcgggcggtgcgctctccgagggccattgcatggagccgaaaagcaaaagcaacagcgaggcagcatggcgatttatcaccttacggcgaaaaccggcagcaggtcgggcggccaatcggccagggccaaggccgactacatccagcgcgaaggcaagtatgcccgcgacatggatgaagtcttgcacgccgaatccgggcacatgccggagttcgtcgagcggcccgccgactactgggatgctgccgacctgtatgaacgcgccaatgggcggctgttcaaggaggtcgaatttgccctgccggtcgagctgaccctcgaccagcagaaggcgctggcgtccgagttcgcccagcacctgaccggtgccgagcgcctgccgtatacgctggccatccatgccggtggcggcgagaacccgcactgccacctgatgatctccgagcggatcaatgacggcatcgagcggcccgccgctcagtggttcaagcggtacaacggcaagaccccggagaagggcggggcacagaagaccgaagcgctcaagcccaaggcatggcttgagcagacccgcgaggcatgggccgaccatgccaaccgggcattagagcgggctggccacgacgcccgcattgaccacagaacacttgaggcgcagggcatcgagcgcctgcccggtgttcacctggggccgaacgtggtggagatggaaggccggggcatccgcaccgaccgggcagacgtggccctgaacatcgacaccgccaacgcccagatcatcgacttacaggaataccgggaggcaatagaccatgaacgcaatcgacagagtgaagaaatccagaggcatcaacgagttagcggagcagatcgaaccgctggcccagagcatggcgacactggccgacgaagcccggcaggtcatgagccagacccagcaggccagcgaggcgcaggcggcggagtggctgaaagcccagcgccagacaggggcggcatgggtggagctggccaaagagttgcgggaggtagccgccgaggtgagcagcgccgcgcagagcgcccggagcgcgtcgcgggggtggcactggaagctatggctaaccgtgatgctggcttccatgatgcctacggtggtgctgctgatcgcatcgttgctcttgctcgacctgacgccactgacaaccgaggacggctcgatctggctgcgcttggtggcccgatgaagaacgacaggactttgcaggccataggccgacagctcaaggccatgggctgtgagcgcttcgatatcggcgtcagggacgccaccaccggccagatgatgaaccgggaatggtcagccgccgaagtgctccagaacacgccatggctcaagcggatgaatgcccagggcaatgacgtgtatatcaggcccgccgagcaggagcggcatggtctggtgctggtggacgacctcagcgagtttgacctggatgacatgaaagccgagggccgggagcctgccctggtagtggaaaccagcccgaagaactatcaggcatgggtcaaggtggccgacgccgcaggcggtgaacttcgggggcagattgcccggacgctggccagcgagtacgacgccgacccggccagcgccgacagccgccactatggccgcttggcgggcttcaccaaccgcaaggacaagcacaccacccgcgccggttatcagccgtgggtgctgctgcgtgaatccaagggcaagaccgccaccgctggcccggcgctggtgcagcaggctggccagcagatcgagcaggcccagcggcagcaggagaaggcccgcaggctggccagcctcgaactgcccgagcggcagcttagccgccaccggcgcacggcgctggacgagtaccgcagcgagatggccgggctggtcaagcgcttcggtgatgacctcagcaagtgcgactttatcgccgcgcagaagctggccagccggggccgcagtgccgaggaaatcggcaaggccatggccgaggccagcccagcgctggcagagcgcaagcccggccacgaagcggattacatcgagcgcaccgtcagcaaggtcatgggtctgcccagcgtccagcttgcgcgggccgagctggcacgggcaccggcaccccgccagcgaggcatggacaggggcgggccagatttcagcatgtagtgcttgcgttggtactcacgcctgttatactatgagtactcacgcacagaagggggttttatggaatacgaaaaaagcgcttcagggtcggtctacctgatcaaaagtgacaagggctattggttgcccggtggctttggttatacgtcaaacaaggccgaggctggccgcttttcagtcgctgatatggccagccttaaccttgacggctgcaccttgtccttgttccgcgaagacaagcctttcggccccggcaagtttctcggtgactgatatgaaagaccaaaaggacaagcagaccggcgacctgctggccagccctgacgctgtacgccaagcgcgatatgccgagcgcatgaaggccaaagggatgcgtcagcgcaagttctggctgaccgacgacgaatacgaggcgctgcgcgagtgcctggaagaactcagagcggcgcagggcgggggtagtgaccccgccagcgcctaaccaccaactgcctgcaaaggaggcaatcaatggctacccataagcctatcaatattctggaggcgttcgcagcagcgccgccaccgctggactacgttttgcccaacatggtggccggtacggtcggggcgctggtgtcgcccggtggtgccggtaaatccatgctggccctgcaactggccgcacagattgcaggcgggccggatctgctggaggtgggcgaactgcccaccggcccggtgatctacctgcccgccgaagacccgcccaccgccattcatcaccgcctgcacgcccttggggcgcacctcagcgccgaggaacggcaagccgtggctgacggcctgctgatccagccgctgatcggcagcctgcccaacatcatggccccggagtggttcgacggcctcaagcgcgccgccgagggccgccgcctgatggtgctggacacgctgcgccggttccacatcgaggaagaaaacgccagcggccccatggcccaggtcatcggtcgcatggaggccatcgccgccgataccgggtgctctatcgtgttcctgcaccatgccagcaagggcgcggccatgatgggcgcaggcgaccagcagcaggccagccggggcagctcggtactggtcgataacatccgctggcagtcctacctgtcgagcatgaccagcgccgaggccgaggaatggggtgtggacgacgaccagcgccggttcttcgtccgcttcggtgtgagcaaggccaactatggcgcaccgttcgctgatcggtggttcaggcggcatgacggcggggtgctcaagcccgccgtgctggagaggcagcgcaagagcaagggggtgccccgtggtgaagcctaagaacaagcacagcctcagccacgtccggcacgacccggcgcactgtctggcccccggcctgttccgtgccctcaagcggggcgagcgcaagcgcagcaagctggacgtgacgtatgactacggcgacggcaagcggatcgagttcagcggcccggagccgctgggcgctgatgatctgcgcatcctgcaagggctggtggccatggctgggcctaatggcctagtgcttggcccggaacccaagaccgaaggcggacggcagctccggctgttcctggaacccaagtgggaggccgtcaccgctgaatgccatgtggtcaaaggtagctatcgggcgctggcaaaggaaatcggggcagaggtcgatagtggtggggcgctcaagcacatacaggactgcatcgagcgcctttggaaggtatccatcatcgcccagaatggccgcaagcggcaggggtttcggctgctgtcggagtacgccagcgacgaggcggacgggcgcctgtacgtggccctgaaccccttgatcgcgcaggccgtcatgggtggcggccagcatgtgcgcatcagcatggacgaggtgcgggcgctggacagcgaaaccgcccgcctgctgcaccagcggctgtgtggctggatcgaccccggcaaaaccggcaaggcttccatagataccttgtgcggctatgtctggccgtcagaggccagtggttcgaccatgcgcaagcgccgccagcgggtgcgcgaggcgttgccggagctggtcgcgctgggctggacggtaaccgagttcgcggcgggcaagtacgacatcacccggcccaaggcggcaggctgaccccccccactctattgtaaacaagacatttttatcttttatattcaatggcttattttcctgctaattggtaataccatgaaaaataccatgctcagaaaaggcttaacaatattttgaaaaattgcctactgagcgctgccgcacagctccataggccgctttcctggctttgcttccagatgtatgctcttctgctcctgcagctaatggatcaccgcaaacaggttactcgcctggggattccctttcgacccgagcatccgtatgatactcatgctcgattattattattatagaagcccccatgaataaatcgctcatcattttcggcatcgtccctgttaacggatccagagaatataaaaagccagattattaatccggcttttttattatttgccgtagagctattcactttaggtttaggatgaaaaaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttactgcgatgagtggcagggcggggcgtaattttttttacgctttacttacgtacttaattcttaaagtatgggcaatcaattggtcgacgataacatcaccgtcgttatcgtcgctttagaataacgttcccaaaatagctcatttccaactggcaactcacaaccaaaaaccgcatttttagtaaatatactcagcaatttgttcaacctgagcatttttcccatttgcaacttgatacaaatatttttagcagcaaattttcctactgccagcttagtttacataaattttgtctgttgacatcttgcacacaataaggtatggcgcatataatgcgatattactaccattaatttactacctagtcattaacgtctcccgccagagaacagttttgaataggtagtcaattttaggtattgaacctgctgtaaatttattaaatcgatgaatttccccgaaatctgctctagcagacttgggttatataccagtaggctcaggtgcaaaacaacaaagcacaaattttacccattaaggatataggcaatctgtcaaatagttgttatctttcttaatacagaggaataatcaacaatatggggcaggtactaactaaagtcctatgcctgtggggcttctgtaaccgacataacctttacgcgttgtcttttaggagtctgttatgaacggtaccatgaattcatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataattaattaacaggagatctacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaagcggccggccgaggagagctagcatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaggccggccgaggagagctagcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaaggcgcgcctgcaggtcgagcggccgctagatctgcatgctctagatttaaatgatatcccggcttatcggtcagtttcacctgatttacgtaaaaacccgcttcggcgggtttttgcttttggaggggcagaaagatgaatgactgtccacgacgctatacccaaaagaaagctagcgttaacagg

SEQ ID NO: 45 - DNA, 13625 bp, Artificial Sequence
  heterologous chimeric plasmid construct
tcgactgtggtctgtctttgttcgctgatctaaacaatacctgaataattgttcatgtgttaatctaaaaatgtgaacaatcgttcaactatttaagacaataccttggaggtttaaaccatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataattaattaacaggagatctacatatgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaagcggccggccgaggagagctagcatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaggccggccgaggagagctagcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaagcggcgcgcctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagaccgagctcgaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgagcatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagctctagag

SEQ ID NO: 46 - DNA, 1920 bp, Artificial Sequence
  Chimeric DNA fragment containing PpetJ promoter linked to E. coli-derived MgsA transferred from plasmid 717 to plasmid 550
tctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagg

SEQ ID NO: 47 - DNA, 1897 bp, Artificial Sequence
  Chimeric DNA fragment containing PpetJ promoter linked to E. coli-derived MgsA transferred from plasmid 718 to plasmid 550
tctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagg

SEQ ID NO: 48 - DNA, 3140 bp, Artificial Sequence
  Chimeric DNA fragment containing E. coli gldA transferred from plasmid 734 to plasmid 728
cccgggattgccataagtaaaggcatcccctgcgtgataagattaccttcactaaaggagcaattatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataacctactccaaactcccggcttgtccgggagtttgaacgcaaaattgcctgtctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagga

SEQ ID NO: 49 - DNA, 3117 bp, Artificial Sequence
  Chimeric DNA fragment containing E. coli gldA transferred from plasmid 734 to plasmid 729
cccgggattgccataagtaaaggcatcccctgcgtgataagattaccttcactaaaggagcaattatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataacctactccaaactcccggcttgtccgggagtttgaacgcaaaattgcctgtctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagga

SEQ ID NO: 50 - DNA, 3176 bp, Artificial Sequence
  Chimeric DNA fragment containing E. coli fucO transferred from plasmid 735 to plasmid 728
cccgggattgccataagtaaaggcatcccctgcgtgataagattaccttcaacaaggagaaggatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaatgcgctgatgaacgctcggttgccgccgggcgttttttattctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagg

SEQ ID NO: 51 - DNA, 3154 bp, Artificial Sequence
  Chimeric DNA fragment containing E. coli fucO transferred from plasmid 735 to plasmid 729
ccccgggattgccataagtaaaggcatcccctgcgtgataagattaccttcaacaaggagaaggatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaatgcgctgatgaacgctcggttgccgccgggcgttttttattctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgatcaaggcttatgccgctttagaggctaatggcaagttgcagccgttcgagtatgatccgggcgctttaggcgccaacgaagttgaaatcgaagttcaatactgcggtgtttgtcattccgacctcagtatgatcaacaatgagtggggtatcagtaactatccgttggttcccggccacgaagttgttggcaccgttgctgctatgggtgagggtgttaatcacgtggaagttggtgacctggttggtttaggctggcacagtggttattgtatgacttgtcactcctgcctgagcggttatcataatttgtgcgctaccgccgagagtactatcgttggtcattatggcggtttcggtgaccgtgtgcgtgctaaaggtgtgtccgttgttaagctgcccaagggtatcgatttggcttccgctggtccgttgttttgcggtggtatcactgtgttttcccccatggttgagttatccctgaaaccgaccgccaaggttgccgttattggtatcggtggtctcggtcacctggccgttcagttcttgcgtgcttggggttgcgaggttaccgctttcactagctccgctcgtaaacagaccgaggttctggagctgggtgcccatcatattttggacagtactaaccccgaagccattgcttccgccgagggtaagttcgattacatcattagtaccgttaatttaaaattggattggaatctgtatatttccactttagccccgcaaggtcactttcatttcgtgggtgttgttctcgaacccctcgacttgaacttgttcccgttgctcatgggtcagcggagtgtgtccgctagtccggttggctccccggctactatcgctactatgctcgatttcgccgttcggcacgatatcaagccggttgttgagcagttctccttcgaccaaattaatgaagccattgctcacttggagtccggtaaggctcactaccgtgtggttttgagtcactccaagaactgaaacgctcggttgccgccgggcgttttttattcctgcagg

SEQ ID NO: 52 - DNA, 3158 bp, Artificial Sequence
  Chimeric DNA fragment containing Akr gene derived from Synechococcus sp. PCC 7002, transferred from plasmid 733 to plasmid 749
cccgggattgccataagtaaaggcatcccctgcgtgataagattaccttcaacaaggagaaggatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaatgcgctgatgaacgctcggttgccgccgggcgttttttattctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgacccgccagaaaaacgagcttatgaaaacaagacaactaggccaaagtgccgtccaaatcaccccgattattctcggtacttggcaagcgggcaagcgcaattgggcggatattgacgaccaagaaattgtggccgggatccgtgccgccgtagatgcaggcattacgaccatcgataccgctgaaatttatggcgatggggattctgaacgtcgggtcgccgaggcgatcgccccccaacgggatcaagtgaccctattaacgaaagtctttgccaatcacctccaccacgaccaggtgatcaccgcctgcgaaaattccctcaacagactccagacagactacatcgatctgtaccaaatccactggccagcgggaacgtggaattctgacctggtgcccatcgctgaaaccatggccgctctgaatcaattgaaagaacagggcaaaattcgcgctattggtgtgtctaatttttccttggcgcaactccaggaagcgatggaacacggccaaatcgatagcattcaaccgccctattctttattttggcgggccattgaacgggaaattcaacctttctgtgcggcccagcagatttcgatcctcgcctattcttccttggcccagggtctactgacggggaaatttggccccgatcaccagtttgcggcgggggatcaccgctcccacaaccgtctttatgctgacccggaaaattaccaacgggtacaaacggccctcggactcctgaaaccgatcgccacgacaaagaattgcaccttggctcaactggcgatcgcctggctgattcggcagccccaaaccaatgccatcgtcggcgcgcgcaatgctcaacaggcgatcgccaatgcccaggccatcgatgtcgagttaacggctaaagatctcgaagccattgaccatatcgggcggacagtaaccgatcctctagacgaaaatccgctcctatggaactggtaagcaccaacgctcggttgccgccgggcgttttttattcctgcagga

SEQ ID NO: 53 - DNA, 3135 bp, Artificial Sequence
  Chimeric DNA fragment containing Akr gene derived from Synechococcus sp. PCC 7002, transferred from plasmid 733 to plasmid 750
cccgggattgccataagtaaaggcatcccctgcgtgataagattaccttcaacaaggagaaggatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaaatgcgctgatgaacgctcggttgccgccgggcgttttttattctagatcaggaattgtaattagaaagtccaaaaattgtaatttaaaaaacagtcaatggagagcattgccataagtaaaggcatcccctgcgtgataagattaccttcagaaaacagatagttgctgggttatcgcagatttttctcgcaaccaaataactgtaaataataactgtctctggggcgacggtaggctttatattgccaaatttcgcccgtgggagaaagctaggctattcaatgtttatggaggactgaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgacccgccagaaaaacgagcttatgaaaacaagacaactaggccaaagtgccgtccaaatcaccccgattattctcggtacttggcaagcgggcaagcgcaattgggcggatattgacgaccaagaaattgtggccgggatccgtgccgccgtagatgcaggcattacgaccatcgataccgctgaaatttatggcgatggggattctgaacgtcgggtcgccgaggcgatcgccccccaacgggatcaagtgaccctattaacgaaagtctttgccaatcacctccaccacgaccaggtgatcaccgcctgcgaaaattccctcaacagactccagacagactacatcgatctgtaccaaatccactggccagcgggaacgtggaattctgacctggtgcccatcgctgaaaccatggccgctctgaatcaattgaaagaacagggcaaaattcgcgctattggtgtgtctaatttttccttggcgcaactccaggaagcgatggaacacggccaaatcgatagcattcaaccgccctattctttattttggcgggccattgaacgggaaattcaacctttctgtgcggcccagcagatttcgatcctcgcctattcttccttggcccagggtctactgacggggaaatttggccccgatcaccagtttgcggcgggggatcaccgctcccacaaccgtctttatgctgacccggaaaattaccaacgggtacaaacggccctcggactcctgaaaccgatcgccacgacaaagaattgcaccttggctcaactggcgatcgcctggctgattcggcagccccaaaccaatgccatcgtcggcgcgcgcaatgctcaacaggcgatcgccaatgcccaggccatcgatgtcgagttaacggctaaagatctcgaagccattgaccatatcgggcggacagtaaccgatcctctagacgaaaatccgctcctatggaactggtaagcaccaacgctcggttgccgccgggcgttttttattcctgcagga

SEQ ID NO: 54 - DNA, 3122 bp, Artificial Sequence
  Akr gene derived from Synechococcus sp. PCC 7002, transferred from plasmid 733 to plasmid 747
ccccgggattgccataagtaaaggcatcccctgcgtgataagattaccttcactaaaggagcaattatggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataacctactccaaactcccggcttgtccgggagtttgaacgcaaaattgcctgtctagagggaattgctctggcaactgattaatccactgagcaacagcccaagacacgcaaacaaaaaccaacgtcttggcgatcgccatcggcaccatgaaaccatcgtaaaagctggggaaagaataaaaaacagtggttcaggaattgcattgccatggccacttcacaaacctagccaattttagcttgaccgcagctttgacagattgtcttttgactttgcctggaccgcctcccataataccttcgcgtcttgaagactttatccttgaaaggagaattcatggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaatattgcacaggtggcaaacgccacctgtttcttacggttttctcgccgccggcactcgacattgccataagtaaaggcatcccctgcgtgataagattaccttcagtttatggaggactgaccatatgacccgccagaaaaacgagcttatgaaaacaagacaactaggccaaagtgccgtccaaatcaccccgattattctcggtacttggcaagcgggcaagcgcaattgggcggatattgacgaccaagaaattgtggccgggatccgtgccgccgtagatgcaggcattacgaccatcgataccgctgaaatttatggcgatggggattctgaacgtcgggtcgccgaggcgatcgccccccaacgggatcaagtgaccctattaacgaaagtctttgccaatcacctccaccacgaccaggtgatcaccgcctgcgaaaattccctcaacagactccagacagactacatcgatctgtaccaaatccactggccagcgggaacgtggaattctgacctggtgcccatcgctgaaaccatggccgctctgaatcaattgaaagaacagggcaaaattcgcgctattggtgtgtctaatttttccttggcgcaactccaggaagcgatggaacacggccaaatcgatagcattcaaccgccctattctttattttggcgggccattgaacgggaaattcaacctttctgtgcggcccagcagatttcgatcctcgcctattcttccttggcccagggtctactgacggggaaatttggccccgatcaccagtttgcggcgggggatcaccgctcccacaaccgtctttatgctgacccggaaaattaccaacgggtacaaacggccctcggactcctgaaaccgatcgccacgacaaagaattgcaccttggctcaactggcgatcgcctggctgattcggcagccccaaaccaatgccatcgtcggcgcgcgcaatgctcaacaggcgatcgccaatgcccaggccatcgatgtcgagttaacggctaaagatctcgaagccattgaccatatcgggcggacagtaaccgatcctctagacgaaaatccgctcctatggaactggtaagcaccaacgctcggttgccgccgggcgttttttattcctgcagga